(19) United States
(12) Patent Application Publication
(10) Pub. No.: US 2023/0093669 A1
(43) Pub. Date: Mar. 23, 2023

(54) ANTIODOR AND ANTIMICROBIAL LAYERS IN ABSORBENT MATERIALS

(71) Applicant: Ascend Performance Materials Operations LLC, Houston, TX (US)

(72) Inventors: Kevin Urman, Houston, TX (US); Scott McCormick, Houston, TX (US); Joseph L. Menner, Houston, TX (US); Kyoung Woo Kim, Houston, TX (US); Gregory Kiggins, Houston, TX (US)

(73) Assignee: Ascend Performance Materials Operations LLC, Houston, TX (US)

(21) Appl. No.: 17/946,493

(22) Filed: Sep. 16, 2022

Related U.S. Application Data
(60) Provisional application No. 63/245,109, filed on Sep. 16, 2021.

(51) Int. Cl. A61L 15/46; A61L 15/22; A61F 13/513

(52) U.S. Cl. CPC A61L 15/46; A61L 15/225; A61F 13/513; A61F 2013/15447

(57) ABSTRACT

An AM/AV material, comprising a topsheet layer comprising fibers comprising a topsheet polymer composition, a backsheet layer comprising fibers comprising a backsheet polymer composition; and an absorbent core configured therebetween, wherein the fibers of at least one of the layers, e.g., the topsheet layer, comprise an AM/AV compound, and wherein the fibers of the topsheet layer demonstrate a rewet value less than 5 g after a first water application and/or a Staph aureus efficacy log reduction greater than 2.6, as measured in accordance with ISO 20743:2013.

### CROSS REFERENCE

This application is related to and claims priority to U.S. Provisional Application No. 63/245,109, filed Sep. 16, 2021, which is incorporated herein by reference.

### FIELD

The present disclosure relates to an absorbent material comprising an AM/AV layer and having (near-permanent) antiodor and/or antiviral and/or antimicrobial (AM/AV) properties. In particular, the present disclosure relates to AM/AV layers made of a polymer composition comprising an AM/AV compound and a polymer that contribute to the unexpected AM/AV properties of the absorbent material.

### BACKGROUND

There is a growing interest in absorbent materials having antiodor and/or antiviral and/or antimicrobial (AM/AV) properties. These products are used to absorb fluids, e.g., bodily fluids.

Conventional absorbent materials, for example absorbent garments, are known. Such materials/garments may include garments worn like underpants for children and adults, garments worn like training pants for toddlers, diapers, as well as feminine hygiene products and adult/child incontinence garments. These absorbent materials are designed to provide the ability to absorb and contain bodily fluids such as urine, feces, and menses.

Absorbent materials or garments typically include at least three layers: a liquid permeable bodyside liner (topsheet); a substantially liquid impermeable outer cover (backsheet) that can be connected to the topsheet in a superposed relation; and an absorbent core located between the backsheet and the topsheet. The topsheet is an interior surface that is configured to contact the wearer's skin during wear. The backsheet is an exterior surface opposite the topsheet that is configured to contact the wearer's clothing during wear.

Some absorbent materials are also known. For example U.S. Pat. No. 8,167,861 discloses a disposable absorbent garment having an elastic inner layer having an interior surface for facing a wearer of the garment, and an exterior surface. The elastic inner layer has an elongate opening therein disposed at least within a crotch region of the garment, and is stretchable in at least a lateral direction of the garment. A backsheet layer of the garment is in opposed relationship with the elastic inner layer and is stretchable in at least the lateral direction of the garment. An absorbent assembly is secured to the elastic inner layer between the elastic inner layer and the backsheet layer and is sized larger than the opening of the elastic inner layer for underlying substantially the entire opening. The absorbent assembly is stretchable in at least the lateral direction of the garment for lateral stretching thereof in response to lateral stretching of the elastic inner layer.

While absorbent materials have been known for many years, the materials used to construct them have continuously evolved as a result of new technologies for formulating and manufacturing such materials.

Some coarse carpet yarns and fabrics with antimicrobial properties are also known. For example, U.S. Pat. No. 4,701,518 discloses an antimicrobial nylon prepared in water with a zinc compound phosphorus compound to form carpet fibers. The process produces nylon fibers for carpets having 18 denier per filament (dpf), and are prepared by conventional melt polymerization. Such carpet fibers typically have average diameters that are well above 30 microns, which are generally unsuitable for next-to-skin applications.

Although some references may disclose absorbent materials, a need exists for absorbent materials that achieve a synergistic combination of absorbency, AM/AV efficacy (including antiodor efficacy), and optionally biocompatibility, e.g. irritation and sensitization, performance.

### SUMMARY

In some cases, the present disclosure relates to an AM/AV material, comprising a topsheet layer comprising fibers comprising a topsheet polymer composition comprising a polymer, e.g., a polyamide, and an AM/AV compound, e.g., a zinc compound; a backsheet layer comprising fibers comprising a backsheet polymer composition; and an absorbent core configured therebetween; wherein the fibers of at least one of the layers comprise an AM/AV compound, and wherein the fibers of at least one of the layers demonstrate a toilet odor reduction greater than 50%, as measured in accordance with ISO 17299-3 (2014) and/or an Escherichia coli efficacy log reduction greater than 4.0, as measured in accordance with ASTM E3160 (2018) and/or a rewet value less than 5 g after a first water application and/or a Staph aureus efficacy log reduction greater than 2.6, as measured in accordance with ISO 20743:2013 and/or efficacy against odor, microbials, bacteria, viruses, fungi, or parasites, or combinations thereof. The polymer compositions may comprise a polymer comprising a thermoplastic polymer, polyester, nylon, rayon, polyamide, polyamide, poly olefin, polyolefin terephthalate, polyolefin terephthalate glycol, co-PET, or polylactic acid, or combinations thereof. The fibers in each of the layers may have an average fiber diameter from 1 micron to 50 microns. The topsheet may have a basis weight ranging from 5 gsm to 40 gsm and/or may be meltblown, spunbond, electrospun, spunlace, or flashspun. The topsheet layer and/or the backsheet layer may have a thickness ranging from 25 microns to 500 microns and/or the backsheet layer has a thickness ranging from 25 microns to 500 microns. The polymer composition(s) may have a hygroscopy absorbance of greater than 1.5 wt. % water, based on the total weight of the polymer. The AM/AV material may further comprise a pad layer comprising a pad polymer composition and configured between the topsheet layer and the absorbent core and/or an acquisition distribution layer comprising an ADL polymer composition and configured between the topsheet layer and the absorbent core and/or an embossed layer comprising a embossed polymer composition and configured between the absorbent core and the backsheet layer. The absorbent core may comprise absorbent material and an AM/AV powder composition comprises an AM/AV compound and a polymer.

The present disclosure also relates to an AM/AV material, comprising a topsheet layer comprising fibers comprising a topsheet polymer composition; a backsheet layer comprising fibers comprising a backsheet polymer composition; and an absorbent core configured therebetween; wherein the AM/AV material comprises an AM/AV powder composition optionally dispersed in the absorbent core and/or in one of the layers, and wherein the AM/AV material demonstrates the aforementioned performance. The AM/AV powder composition comprises an AM/AV compound, e.g., a zinc compound, and a polymer, e.g., a polyamide. The polymer compositions may comprise a polymer comprising a thermoplastic polymer, polyester, nylon, rayon, polyamide, polyamide, poly olefin, polyolefin terephthalate, polyolefin terephthalate glycol, co-PET, or polylactic acid, or combinations thereof.

The present disclosure also relates to a process for making an AM/AV material, comprising the steps of: providing a topsheet layer comprising fibers comprising a topsheet polymer composition; a backsheet layer comprising fibers comprising a backsheet polymer composition; and an absorbent core; wherein the fibers of at least one of the layers comprise an AM/AV compound configuring the absorbent core between the topsheet layer and the backsheet layer; wherein the fibers of at least one of the layers demonstrate the aforementioned performance. In some cases, none of the layers are treated to improve initial hygroscopicity and/or hydrophilicity.

### DETAILED DESCRIPTION

### Introduction

Absorbent materials are generally known as materials, e.g., configurations of fabrics or layers, that are made from at least one material that has high absorbency. The absorbent materials are typically designed to absorb, for example, fluids, liquids, loose solids, and/or slurries (individually or collectively referred to herein as fluids or bodily fluids) in many applications. In many cases, the fluids are fluids excreted by the human or animal body such as urine, feces, and menses. Exemplary absorbent materials include products or garments for use in the incontinence industry, products used for feminine hygiene, hospital pads, and pet pads.

Conventional absorbent material suffer from many shortcomings, not the least of which is that they are designed to simply absorb and hold fluids. This is disadvantageous because of the many deleterious effects that accompany these fluids, examples of which include bacterial growth, microbial activity, unpleasant odor, and generally unhealthy environments.

It has now been discovered that the employment of the disclosed antiodor, antifungal, antimicrobial, and/or antiviral (“AM/AV”) compounds in absorbent materials, e.g., as an AM/AV layer in an absorbent material configuration or as an AM/AV powder disposed in one or more of the layers of the absorbent material, beneficially provides for a synergistic combination of absorbency and AM/AV properties in the resultant absorbent material. The AM/AV materials demonstrate efficacy against odor, microbials, bacteria, viruses, fungi, or parasites, or combinations thereof. The addition of the AM/AV compounds are particularly advantageous when utilized in conjunction with fabrics made of hygroscopic polymers, e.g., polyamides (collectively AM/AV (polymer) compositions). The inventors have found that the negative effects of bodily fluids may be effectively countered by treatment with the AM/AV compounds. In some particular cases, and without being bound by theory, it is postulated that that the hygroscopic nature of some polymers pulls in fluids and allows the AM/AV compounds to interact with the fluids to counter/combat them, so as to retard or eliminate the accompanying odor, fungi, microbials, and/or viruses and promote a more healthy environment. In some cases, the use of the aforementioned AM/AV compounds or layers advantageously serves to prevent unpleasant odors from emanating from the absorbent materials.

Conventionally, some olefin fabrics that are employed in absorbent materials must be treated in an effort to make these fabrics more hygroscopic/hydrophilic. For example, complex lotions are applied to conventional olefin topsheet layers. Advantageously, the fabrics discussed herein do not require such treatment. And as a result, these costly treatment steps can be reduced or eliminated, which, in turn, provides for significant process efficiencies.

Importantly, although some conventional adhesives and adhesive application methods are known, problems persist with regard to inter-layer adhesion. And these problems have now been found to be exacerbated with some particular layer materials, e.g., the AM/AV fabrics. For example, proper contact and adhesion is not obtained. Also, many conventional adhesive and application methods may have deleterious effects on the AM/AV properties of the AM/AV fabrics/layers due to chemical interaction with the components thereof.

The inventors have found that with the use of AM/AV layers, specific bonding methods/devices may be preferably employed to ensure proper contact between the layers and to also avoid deleterious effects on the AM/AV properties of the AM/AV layers. In particular, some adhesives have been found to demonstrate particularly good performance when employed with the AM/AV layers. Also, specific application/production methods have been unexpectedly efficient and effective.

In addition to the hygroscopic benefits, the disclosed AM/AV polymer compositions has been found to be unexpectedly compliant, soft feeling (low denier/small diameter), and nonirritating to the wearer's skin. This is particularly beneficial in next-to-skin applications. It was found that the disclosed AM/AV compounds, e.g., zinc compounds, are particularly advantageous for this reason (vs. silver or other less skin-friendly compounds).

The AM/AV polymer compositions, which include both the AM/AV compounds and the polymer components are discussed in detail below.

The present disclosure relates to absorbent, AM/AV materials having AM/AV properties. In particular, the present disclosure provides absorbent materials that include one or more AM/AV absorbent materials as described herein. As discussed above, absorbent materials are designed to protect a user from (prolonged) touching of fluids, e.g., bodily fluids, including liquids, particulate matter and accompanying airborne pathogens, e.g., bacteria and/or viruses.

The AM/AV material may be a fabric or mat or collection of fibers, and the AM/AV material, in some cases, may contain multiple layers (although single layer materials are contemplated). In some embodiments, the disclosure relates to AM/AV materials that have beneficial performance properties, e.g., those discussed herein. The absorbent AM/AV materials of the present disclosure may be utilized in a variety of industries, including incontinence, feminine hygiene, public safety, healthcare, pet, and textile industries. Exemplary AM/AV materials include (but are not limited to) diapers, feminine hygiene products, and pet pads.

The AM/AV material, in some cases, may comprises a topsheet layer. Generally, the topsheet layer may be a liquid permeably bodyside layer. The topsheet may provide an interior surface that contacts the wearer's skin during wear. The topsheet layer comprises fibers that are made from or that comprise a topsheet polymer composition. The topsheet polymer composition may comprise a polymer and an AM/AV compound, e.g., a polyamide and a zinc compound, which are discussed in detail below.

The AM/AV material may comprise a backsheet layer. Generally, the backsheet layer may be a substantially (or totally permeable layer). The backsheet may provide an interior surface that contacts the wearer's skin during wear. The backsheet, in some cases, may be an exterior surface opposite the topsheet that is configured to contact the wearer's clothing during wear. In some cases, the backsheet may have layer that is outward of it, e.g., a textured layer, in such a case the textured layer may contact the wearer's clothing during wear (or faces the air if a diaper is worn without outer clothing). In other applications, where the AM/AV material is not worn per se (for example in a pet pad), the backsheet layer may be exposed (to air or to the ground) or the textured layer outward of the backsheet layer may be exposed. The backsheet layer comprises fibers that are made from or that comprise a backsheet polymer composition. The backsheet polymer composition may be different from the topsheet polymer composition (although it is contemplated that both compositions may be the same).

In some embodiments, the AM/AV material may comprise an absorbent core configured between the topsheet layer and the backsheet layer. In some cases, the absorbent cores is immediately adjacent the topsheet and backsheet layers. In some case, the absorbent core is adjacent other layers but is still in between the topsheet and backsheet layers.

The fibers of at least one of the layers of the AM/AV material comprise an AM/AV compound, and the AM/AV compound contributes to the AM/AV properties of the AM/AV material. For example, at least one of the layers (or the fibers thereof) demonstrates a toilet odor reduction greater than 50%, e.g., greater than 60%, greater than 70%, or greater than 80%, as measured in accordance with ISO 17299-3 (2014). Toilet odor may be tested using specific test chemicals, e.g., ammonia, acetic acid, isovaleric acid, hydrogen sulfide, indole, and/or nonenal. At least one of the layers (or the fibers thereof) demonstrates the toilet odor reduction for one or more of these test chemicals.

In some cases, the AM/AV materials or the layers thereof are not harmful to the wearer/user. For example, the AM/AV materials or the layers thereof may be tested to assess in vitro cytotoxicity of the materials (e.g., according to ISO 10993-5:2009) and/or to assess the potential of the materials to produce skin irritation (e.g., according to ISO 10993-10:2010). In some embodiments, the AM/AV materials or the layers thereof, as described herein, adequately passes such testing.

In some cases, the disclosure relates to an AM/AV material that comprises the topsheet layer and the backsheet layer and the absorbent core. The AM/AV material may further comprise an AM/AV powder composition. The AM/AV powder composition may contribute to the AM/AV performance of the AM/AV material. For example, the AM/AV material may demonstrate a toilet odor reduction greater than 50%, as measured in accordance with ISO 17299-3 (2014). In some cases, the AM/AV powder composition comprises an AM/AV compound and optionally a polymer, e.g., a polyamide and optionally a zinc compound. In some embodiments, the AM/AV powder composition is dispersed in the absorbent core and/or in one of the layers, e.g., the topsheet or backsheet layers or other third layers. The AM/AV compound may contribute to the AM/AV performance.

In some embodiments, the described AM/AV materials demonstrate antimicrobial and/or antiviral properties. In particular, the antimicrobial and/or antiviral properties may be the result of forming the AM/AV materials from the polymer compositions described herein. As discussed in further detail below, the polymer compositions exhibit a relatively high metal retention rate. As a result, the AM/AV materials formed from the polymer composition may demonstrate permanent, e.g., near-permanent, AM/AV properties. In particular, the metal retention rate may allow the AM/AV materials to be washed and/or reused without reduced effectiveness, e.g., without reduced AM/AV properties. This is a marked improvement over conventional materials, which typically lose AM/AV effect with time, e.g., as a result of static decay.

The AM/AV materials of the present disclosure, unlike conventional materials, advantageously utilize one or more layers that, in addition to relying on physical filtration properties, also provide AM/AV properties, e.g., pathogen-destroying properties. Stated another way, the disclosed AM/AV materials not only protect by limiting pathogen intake, they also destroy pathogens via contact with the AM/AV layer(s) before the pathogens have a chance to enter or contact the body. The AM/AV properties are made possible, at least in part, by the composition of the fibers that make up the layers. At least one of the layers contains a polymer component along with an AM/AV compound, e.g., zinc and/or copper, which in some cases, is embedded in the polymer structure (but may not be a component of a polymerized co-polymer). The presence of the AM/AV compound in the polymers of the fibers provides for the pathogen-destroying properties. As a result, the disclosed items prevent growth or transmission of pathogens from contact that otherwise would allow the pathogen to spread. Importantly, because the AM/AV compound may be embedded in the polymer structure, the AM/AV properties are durable, and are not easily worn or washed away. Thus, the AM/AV materials disclosed herein achieve a synergistic combination of AM/AV efficacy and absorbent performance (and optionally biocompatibility, e.g. irritation and sensitization, performance). In contrast, conventional configurations that employ no AM/AV compound (or that do not meet the disclosed physical characteristic limits, e.g., basis weight or fiber diameter) do not and cannot provide the aforementioned synergistic combination of performance features.

In some embodiments, the disclosure relates to an AM/AV material, comprising the topsheet layer, the backsheet layer, the absorbent core; and an adhesive disposed between at least two of the layers. Beneficially, in view of the adhesive selection and application, the bond strength between the adhered layers is high. In addition, the adhesive does not deleteriously affect the AM/AV properties of the layers, which results in a synergistic combination of performance features.

The selection of the adhesive may contribute to the synergistic combination of features. In some cases, the adhesive may comprise a polyolefin, rubber, epoxy, urethane, urea, acrylate, or acrylic, or a combination thereof. Other suitable adhesive include, but are not limited to a water-based acrylate adhesive, an oil-based adhesive, a polyolefin-based adhesive, an acrylate-based contact adhesive, a polyurethane-based adhesive, a kraton-based adhesive, a styrene-butadiene adhesive, vinyl acetate based adhesive, or siloxane based adhesive, or a combination thereof. Specific commercial products include Rhodotac 315 from Rhodia Ltd. (Manchester, United Kingdom).

The inventors have found that particular loading rates contribute to the aforementioned features. In addition, the particular loading rates contribute to weight reduction (and ultimately cost reduction), while maintaining good or acceptable performance. In some embodiments, the adhesive is applied at a loading rate ranging from 0.5 gsm to 30 gsm, e.g., from 0.5 gsm to 25 gsm, from 0.5 gsm to 20 gsm, from 1 gsm to 25 gsm, from 1 gsm to 15 gsm, from 2 gsm to 12 gsm, or from 3 gsm to 10 gsm. In terms of lower limits, the adhesive is applied at a loading rate greater than 0.5 gsm, greater than 1 gsm, greater than 2 gsm, greater than 3 gsm, greater than 5 gsm, or greater than 10 gsm. In terms of upper limits, the adhesive is applied at a loading rate less than 30 gsm, e.g., less than 25 gsm, less than 20 gsm, less than 17 gsm, less than 15 gsm, less than 12 gsm, less than 10 gsm, less than 8 gsm, or less than 5 gsm.

The arrangement or configuration of the layers and the adhesive may vary widely. Generally, at least one of the layers that has AM/AV properties can be effectively adhered to another layer. Some exemplary configurations are provided below. These are not meant to limit the configurations. All of the various layers have the potential to contain the AM/AV compound. And any such layers may be configured and adhered to another layer, whether the other layer comprises the AM/AV compound or not. In some cases, the topsheet layer comprises the AM/AV compound and, as such, has AM/AV properties. AM/AV properties are disclosed herein.

In some embodiments, the adhesive is disposed between the topsheet layer, which optionally comprises the AM/AV compound, and the absorbent core. In some cases, the adhesive is disposed between the absorbent core and the backsheet, which optionally comprises the AM/AV compound. In some embodiments, the AM/AV material further comprises an acquisition distribution layer (ADL) comprising an ADL polymer composition. The ADL may be configured between the topsheet layer and the absorbent core. The ADL is described in more detail below.

In some embodiments, the adhesive is disposed between the topsheet layer, which optionally comprises the AM/AV compound, and the ADL. In some cases, the adhesive is disposed between the acquisition distribution layer and the absorbent core. In some embodiments, the AM/AV material further comprises a pad layer comprising a pad polymer composition. The pad layer may be configured between the topsheet layer and the absorbent core. The pad layer is described in more detail below.

In some embodiments, the adhesive is disposed between the topsheet, which optionally comprises the AM/AV compound, and the pad layer. In some cases, the adhesive is disposed between the pad layer and the absorbent core. In some embodiments, the AM/AV material further comprises an embossed layer comprising a embossed polymer composition. The embossed layer may be configured between the absorbent core and the backsheet layer.

In some embodiments, the adhesive is disposed between the absorbent core and the embossed layer. In some cases, the adhesive is disposed between the embossed layer and the backsheet layer, which optionally comprises the AM/AV compound. In some cases, the absorbent core layer and/or the backsheet layer comprise the AM/AV compound and, as such, have AM/AV properties.

The disclosure also relates to a process for making an AM/AV material. The process comprises the steps of providing the aforementioned topsheet layer, backsheet layer, and absorbent core. The process further comprises the step of configuring the absorbent core between the topsheet layer and the backsheet layer. The process comprises the step of applying the aforementioned adhesive between at least two of the layers to form the AM/AV material. As a result of the specific adhesive and application parameters, the bond strength between the adhered layers is high, as noted above and the adhesive does not deleteriously affect the AM/AV properties of the layers. As noted above, the application of the adhesive, as described herein, provides for a synergistic combination of performance features.

In some cases, the applying is conducted at a line speed ranging from 100 feet per minute to 2500 feet per minute, e.g., from 150 fpm to 2500 fpm, from 300 fpm to 2000 fpm, from 300 fpm to 2000 fpm, from 500 fpm to 1500 fpm, or from 700 fpm to 1200 fpm. In terms of lower limits, the applying may be conducted at a line speed greater than 100 fpm, e.g., greater than 150 fpm, greater than 300 fpm, greater than 400 fpm, greater than 500 fpm, greater than 600 fpm, greater than 700 fpm, greater than 1000 fpm, or greater than 1200 fpm. In terms of upper limits, the applying may be conducted at a line speed less than 2500 fpm, e.g., less than 2200 fpm, less than 2100 fpm, less than 2000 fpm, less than 1700 fpm, less than 1500 fpm, less than 1200 fpm, or less than 1000 fpm. The aforementioned line speeds have been determined to advantageously maximize throughput and/or provide the appropriate open time (time between application and adhesion) for the disclosed adhesives.

In some cases, the applying may be conducted via spray and/or contact rolling. In some embodiments, the applying may be conducted via sonic bonding. The process may be conducted using the ranges and limits for adhesive loading rates, as discussed above. In some embodiments, the applying is conducted using an predetermined adhesive pattern.

Further, the inclusion of at least one layer that comprises polyamide polymer has been shown to increase overall hydrophilicity and/or hygroscopy of the AM/AV materials, which works synergistically with the AM/AV compound to destroy pathogens. For example, it is theorized that a polymer of increased hydrophilicity and/or hygroscopy both may better attract liquid and/or capture media that carry microbials and/or viruses, e.g., waste, and may also absorb more moisture, e.g., from the air, and that the increased moisture content allows the polymer composition and the AM/AV compound to more readily destroy, limit, reduce, or inhibit infection and/or pathogenesis of a microbe or virus. For example, the moisture may dissolve an backsheet layer, e.g., capsid, of a virus, exposing the genetic material, e.g., DNA or RNA, of the virus.

In addition, in some cases, the disclosed AM/AV materials may contain little or no reinforcement material, e.g., glass- and/or carbon fibers, (carbon) nanotubes, particulate fillers, such as mineral fillers based on natural and/or synthetic layer silicates, talc, mica, silicate, quartz, titanium dioxide, wollastonite, kaolin, amorphous silicic acids, magnesium carbonate, magnesium hydroxide, chalk, lime, feldspar, barium sulphate, solid or hollow glass balls or ground glass, permanently magnetic or magnetizable metal compounds and/or alloys and/or combinations thereof, and also combinations thereof. In some cases, the disclosed AM/AV materials comprise less than 50 wt % of these materials, e.g., less than 25 wt %, less than 10 wt %, less than 5 wt %, less than 1 wt %, less than 5000 wppm, or less than 1000 wppm.

The composition of the fibers, fabrics, and layers is discussed in more detail herein. And the methods of producing the fibers, fabrics, layers, and materials, e.g., spunbonding, spun lace, melt blowing, electrospinning, inter alia, are discussed in more detail herein. Other production processes are contemplated, including textile spinning and weaving.

As noted above, the present disclosure provides novel compositions and configurations for AM/AV materials.

In particular, the AM/AV materials may comprise multiple layers. At least one of the layers demonstrate the AM/AV properties. That is to say, at least one of the layers has the ability to destroy pathogens, odors, fungi, etc. that come into contact with the layer. As a result, the AM/AV materials provide for the aforementioned benefits. As is discussed in detail below, the AM/AV properties of the AM/AV materials may be derived from the use of a polymer composition demonstrating AM/AV properties. The present disclosure encompasses several configurations of the AM/AV materials. In addition to the AM/AV properties, the configurations exhibit varying levels of absorbent performance characteristics. As such, the AM/AV materials of the present disclosure may be configured to satisfy various industry standards. In some embodiments, for example, the AM/AV materials (diaper products) described herein have an absorbency of greater than 5 ml of absorbed material per diaper, e.g., greater than 10 ml, greater than 25 ml, greater than 50 ml, greater than 75 ml, greater than 100 ml, greater than 200 ml, greater than 300 ml, or greater than 500 ml. In terms of ranges, the AM/AV materials (diaper products) described herein have an absorbency ranging from 5 ml to 2000 ml of absorbed material per diaper, e.g., from 10 ml to 1000 ml, from 25 ml to 500 ml, from 50 ml to 500 ml, or from 100 ml to 300 ml

In some cases, the disclosure relates to the material from which the layers are formed, e.g., to the fibers or fabrics. The fibers or fabrics may be produced as discussed herein and collected in bulk, e.g., in high quantities on rolls. The rolled fabric may then be further processed to produce the disclosed layers.

### Absorbent Material Configurations

The AM/AV materials of the present disclosure may include multiple layers. In particular, the AM/AV materials comprise the topsheet layer and the backsheet, e.g., a far backsheet layer. In some embodiments, the AM/AV materials include an additional third layer, e.g., an absorbent core, that is outward of the topsheet layer. In some cases, the layers are arranged such that at least a portion of the third layer is disposed between the topsheet layer and the backsheet layer, e.g., the third layer is sandwiched between the topsheet and backsheet layers. In some embodiments, the layers of the AM/AV materials are arranged such that at least a portion of the topsheet layer is in contact with the third layer. In some embodiments, the layers of the AM/AV materials are arranged such that at least a portion of the backsheet layer is in contact with the third layer. In some cases, the topsheet layer, the backsheet layer, and the third layer are (at least substantially) coextensive.

In some embodiments, the AM/AV materials may comprise additional layers, which may be similar to or distinct from each of the topsheet, backsheet, and third layers. Said another way, in some cases, other layers may also be included in the AM/AV materials. In embodiments with additional layers, the backsheet layer may not necessarily be in direct contact with the other layers. That is to say, “disposed between,” e.g., the third layer is disposed between the topsheet layer and the backsheet layer) does not necessarily mean “in contact with.” In some cases, the layers may be made up of sublayers, e.g., multiple sublayers may be combined to form one of the primary layers, e.g., the third layer may include multiple layers.

Importantly, at least one of the layers, e.g., the topsheet layer, may be comprised of fibers or fabrics that have the AM/AV properties discussed herein. As such, these layers have the capability to kill pathogens and/or odors that contact the layer(s). For example, the topsheet layer may be constructed of AM/AV fibers, and this layer may destroy pathogens that pass through the backsheet or third layers, thus providing superior AM/AV performance. It is contemplated that any of the layers disclosed herein may beneficially be made from the polymer compositions disclosed herein, and as such will have the aforementioned AM/AV benefits.

As used herein, the term “coextensive” refers to a relationship between two or more layers such that the surface areas of adjacent or parallel faces of the layers are aligned with one another with little or no overhang (of at least one of the areas or layers). In some cases the extents of the areas or faces are within 90% of one another. For example, two or more layers are coextensive if the surface areas of adjacent or parallel faces of the layers are within 90%, within 92%, within 94%, within 96%, or within 98% of one another. The term “coextensive” can also refer to a relationship between two or more layers such that the lengths of the layers are within 90% of one another. For example, two or more layers are coextensive if the lengths of the layers are within 90%, within 92%, within 94%, within 96%, or within 98% of one another. The term “coextensive” can also refer to a relationship between two or more layers such that the widths of the layers are within 90% of one another. For example, two or more layers are coextensive if the widths of the layers are within 90%, within 92%, within 94%, within 96%, or within 98% of one another.

In some embodiments, the configuration/arrangement/attachment of the various layers may vary widely, and methods for this are known. For example, layers may be adhered, sewn, tacked, or stapled to one another. Physical and adhesive attachments are contemplated.

In some embodiments, the various layers may be formed directly on other layers. For example, the topsheet layer may comprise a nonwoven polyamide fabric, and an intermediate, third layer may comprised a plurality of polyamide nanofibers, which are blown directly on a surface of the topsheet layer. In this way, the topsheet and the third layer may be (substantially) contiguous.

Some coarse carpet yarns and fabrics with antimicrobial properties are also known. For example, U.S. Pat. No. 4,701,518 discloses an antimicrobial nylon prepared in water with a zinc compound phosphorus compound to form carpet fibers. The process produces nylon fibers for carpets having 18 denier per filament (dpf), and are prepared by conventional melt polymerization. Such carpet fibers typically have average diameters that are well above 30 microns, which are generally unsuitable for next-to-skin applications. Furthermore, the conventional additives added to polymer compositions to impart antimicrobial properties in the synthetic fibers made therefrom have been found to reduce the relative viscosity in the polymer compositions. This reduced relative viscosity produces further difficulty in producing synthetic fibers from the polymer composition, e.g., increased difficulty in extruding the polymer composition.

### Topsheet Layer

The disclosed AM/AV materials include a topsheet layer, e.g., an inner layer. Because the topsheet layer may be adjacent to a user's body, the disclosed topsheet layers may provide comfort and/or to fit to the user, for example due to the softness or formability of the layers, e.g., due to the characteristics of the fabric such as fiber diameter or denier, which may provide the softness. The topsheet layer may be constructed of AM/AV fibers and/or fabrics, and as such, may impart AM/AV capabilities thereto. As a result, the topsheet layer may prevent transmission of pathogens from contact that otherwise would allow the pathogen to spread or to pass through the material to the wearer.

The topsheet layer may be composed of first fibers or a first fabric. In some cases, the first fabric is a polymer fabric, e.g., a polyamide fabric. The structure of the first fabric is not particularly limited. In some embodiments, the fabric is a nonwoven fabric. In some embodiments, the first fabric is a woven fabric. In some embodiments, the first fabric is a knit fabric. For example, the first fabric may be composed of a spunbond fabric, a meltblown fabric, or a flashspun fabric, although other formation methods are contemplated. In some cases, the first fabric comprises polyamide fibers, e.g., polyamide microfibers or polyamide nanofibers.

Generally speaking, the differences in production method have been found to be important. For example, because of the nature of the respective processing, the characteristics of the various fabrics have been found to be unexpectedly beneficial when acting as specific layers. In some cases, meltblown fabrics are beneficial because they advantageously provide softness, which beneficially improves performance in next-to-skin applications. As another example, the polyamide polymer composition may provide hydrophilic and/or hygroscopic features, which are beneficial for the reasons discussed herein. In some cases, spunbond fabrics may be suitable for backsheet layers, e.g., topsheet layers and/or backsheet layers, due to the larger filament size. This may work synergistically with the other layers to provide for the aforementioned combinations of performance features. Also, the integrity of spunbond fabrics, especially those fabrics that have been calendered may contribute to the overall strength, wear, and durability of the resultant materials. For example, the outer polyamide fabric may be composed of a spunbond fabric, a meltblown fabric, electrospun fabric, spunlace fabric, or a flashspun fabric.

The composition of the topsheet layer, e.g., the composition of the first fabric and/or the fibers thereof, may vary widely. In some embodiments, the first fabric and/or the fibers thereof are made from and/or comprises the polymer composition, e.g., the polyamide composition, which is discussed in detail below. The polyamide composition comprises a polymer and an AM/AV compound, and in some cases, the AM/AV compound provided for the AM/AV benefits. In some cases, the first fabric is a polymer, e.g., polyamide, fabric made from the polymer compositions described herein.

In some cases, the first fabric is a polyamide fabric. Examples of suitable polyamides include PA-4T/4I, PA-4T/6I, PA-5T/5I, PA-6, PA6,6, PA6,6/6, PA6,6/6T, PA-6T/6I, PA-6T/6I/6, PA-6T/6, PA-6T/6I/66, PA-6T/MPMDT, PA-6T/66, PA-6T/610, PA-10T/612, PA-10T/106, PA-6T/612, PA-6T/10T, PA-6T/10I, PA-9T, PA-10T, PA-12T, PA-10T/10I, PA-10T/12, PA-10T/11, PA-6T/9T, PA-6T/12T, PA-6T/10T/6I, PA-6T/6I/6, or PA-6T/61/12, or copolymers thereof, or blends, mixtures or combinations thereof. In particular, the first polyamide fabric may be composed of a polymer composition as described herein.

In some cases, the first fabric is conventional polymer fabric. For example, the first fabric may comprise a fabric made from polyester, nylon, rayon, polyamide 6, polyamide 6,6, polyethylene (PE), polypropylene (PP), polyethylene terephthalate (PET), polyethylene terephthalate glycol (PETG), co-PET, polybutylene terephthalate (PBT) polylactic acid (PLA), and polytrimethylene terephthalate (PTT).

The basis weight of the topsheet layer, e.g., the basis weight of the first fabric) may vary widely. In one embodiment, the topsheet layer has a basis weight from 2 g/m² to 40 g/m², e.g., 5 g/m² to 40 g/m², from 2 g/m² to 30 g/m², from 5 g/m² to 28 g/m², from 5 g/m² to 26 g/m², from 5 g/m² to 25 g/m², from 5 g/m² to 24 g/m², from 5 g/m² to 22 g/m², from 2 g/m² to 15 g/m², 6 g/m² to 30 g/m², from 6 g/m² to 28 g/m², from 6 g/m² to 26 g/m², from 6 g/m² to 24 g/m², from 6 g/m² to 22 g/m², 7 g/m² to 30 g/m², from 7 g/m² to 28 g/m², from 7 g/m² to 26 g/m², from 7 g/m² to 24 g/m², from 7 g/m² to 22 g/m², 8 g/m² to 30 g/m², from 8 g/m² to 28 g/m², from 8 g/m² to 26 g/m², from 8 g/m² to 24 g/m², from 8 g/m² to 22 g/m², 9 g/m² to 30 g/m², from 9 g/m² to 28 g/m², from 9 g/m² to 26 g/m², from 9 g/m² to 24 g/m², from 9 g/m² to 22 g/m², from 15 g/m² to 25 g/m², or from 10 g/m² to 20 g/m².

In terms of lower limits, the basis weight of the topsheet layer, e.g., of the first fabric, may be greater than 5 g/m², e.g., greater than 6 g/m², greater than 7 g/m², greater than 8 g/m², greater than 9 g/m², or greater than 10 g/m². In terms of upper limits, the basis weight of the topsheet layer, e.g., of the first fabric, may be less than 40 g/m², e.g., less than 35 g/m², less than 30 g/m², less than 28 g/m², less than 26 g/m², less than 25 g/m², less than 24 g/m², less than 22 g/m², or less than 20 g/m². In some cases, the basis weight of the topsheet layer (e.g., of the first fabric) may be about 8 g/m², about 9 g/m², about 10 g/m², about 11 g/m², about 12 g/m², about 13 g/m², about 14 g/m², about 15 g/m², about 16 g/m², about 17 g/m², about 18 g/m², about 19 g/m², about 20 g/m², about 21 g/m², or about 22 g/m², or a basis weight therebetween.

As used herein, “greater than” and “less than” limits may also include the number associated therewith. Stated another way, “greater than” and “less than” may be interpreted as “greater than or equal to” and “less than or equal to.” It is contemplated that this language may be subsequently modified in the claims to include “or equal to.” For example, “greater than 4.0” may be interpreted as, and subsequently modified in the claims as “greater than or equal to 4.0.”

In some cases, the layer(s) mentioned herein may be knit fabrics (not nonwoven). In some embodiments, e.g., when the topsheet layer is a knit fabric, the basis weight may be significantly higher. For example, the topsheet layer may have a basis weight from 5 g/m² to 200 g/m², e.g., from 50 g/m² to 200 g/m², 110 g/m² to 200 g/m², from 120 g/m² to 190 g/m², from 130 g/m² to 180 g/m², from 140 g/m² to 170 g/m², or from 150 g/m² to 160 g/m². In terms of lower limits, the basis weight of the topsheet layer may be greater than 5 g/m², e.g., greater than 50 g/m², greater than 110 g/m², greater than 120 g/m², greater than 130 g/m², greater than 140 g/m², or greater than 150 g/m². In terms of upper limits, the basis weight of the topsheet layer may be less than 200 g/m², e.g., less than 190 g/m², less than 180 g/m², less than 170 g/m², or less than 160 g/m². In some instances the other layers discussed herein may be knit fabrics and may have these basis weight ranges and limits.

In some embodiments, the topsheet layer comprises a plurality of fibers having an average fiber diameter less than 50 microns, e.g., less than 45 microns, less than 40 microns, less than 35 microns, less than 30 microns, less than 25 microns, less than 20 microns, less than 15 microns, less than 10 microns, or less than 5 microns. In terms of lower limits, the plurality of fibers may have an average fiber diameter greater than 1 micron, e.g., greater than 1.5 microns, greater than 2 microns, greater than 2.5 microns, greater than 5 microns, or greater than 10 microns. In terms of ranges, the plurality of fibers may have an average fiber diameter from 1 micron to 50 microns, e.g., from 1 micron to 45 microns, from 1 micron to 40 microns, from 1 micron to 35 microns, from 1 micron to 30 microns, from 1 micron to 20 microns, from 1 micron to 15 microns, from 1 micron to 10 microns, from 1 micron to 5 microns, from 1.5 microns to 25 microns, from 1.5 microns to 20 microns, from 1.5 microns to 15 microns, from 1.5 microns to 10 microns, from 1.5 microns to 5 microns, from 2 microns to 25 microns, from 2 microns to 20 microns, from 2 microns to 15 microns, from 2 microns to 10 microns, from 2 microns to 5 microns, from 2.5 microns to 25 microns, from 2.5 microns to 20 microns, from 2.5 microns to 15 microns, from 2.5 microns to 10 microns, from 2.5 microns to 5 microns, from 5 microns to 45 microns, from 5 microns to 40 microns, from 5 microns to 35 microns, from 5 microns to 30 microns, from 10 microns to 45 microns, from 10 microns to 40 microns, from 10 microns to 35 microns, from 10 microns to 30 microns. In some cases, fibers of this size may be referred to as microfibers.

In some embodiments, the topsheet layer comprises a plurality of fibers having an average fiber diameter less than 1 micron, e.g., less than 0.9 microns, less than 0.8 microns, less than 0.7 microns, less than 0.6 microns, less than 0.5 microns, less than 0.4 microns, less than 0.3 microns, less than 0.2 microns, less than 0.1 microns, less than 0.05 microns, less than 0.04 microns, or less than 0.03 microns. In terms of lower limits, the average fiber diameter of the plurality of fibers may be greater than 1 nanometer, e.g., greater than 10 nanometers, greater than 25 nanometers, or greater than 50 nanometers. In terms of ranges, the average fiber diameter of the plurality of fibers may be from 1 nanometer to 1 micron, e.g., from 1 nanometer to 0.9 microns, from 1 nanometer to 0.8 microns, from 1 nanometer to 0.7 microns, from 1 nanometer to 0.6 microns, from 1 nanometer to 0.5 microns, from 1 nanometer to 0.4 microns, from 1 nanometer to 0.3 microns, from 1 nanometer to 0.2 microns, from 1 nanometer to 0.1 microns, from 1 nanometer to 0.05 microns, from 1 nanometer to 0.04 microns, from 1 nanometer to 0.3 microns, from 10 nanometers to 1 micron, from 10 nanometers to 0.9 microns, from 10 nanometers to 0.8 microns, from 10 nanometers to 0.7 microns, from 10 nanometers to 0.6 microns, from 10 nanometers to 0.5 microns, from 10 nanometers to 0.4 microns, from 10 nanometers to 0.3 microns, from 10 nanometers to 0.2 microns, from 10 nanometers to 0.1 microns, from 10 nanometers to 0.05 microns, from 10 nanometers to 0.04 microns, from 10 nanometers to 0.03 microns, from 25 nanometers to 1 micron, from 25 nanometers to 0.9 microns, from 25 nanometers to 0.8 microns, from 25 nanometers to 0.7 microns, from 25 nanometers to 0.6 microns, from 25 nanometers to 0.5 microns, from 25 nanometers to 0.4 microns, from 25 nanometers to 0.3 microns, from 25 nanometers to 0.2 microns, from 25 nanometers to 0.1 microns, from 25 nanometers to 0.05 microns, from 25 nanometers to 0.04 microns, from 25 nanometers to 0.03 microns, from 50 nanometers to 1 micron, from 50 nanometers to 0.9 microns, from 50 nanometers to 0.8 microns, from 50 nanometers to 0.7 microns, from 50 nanometers to 0.6 microns, from 50 nanometers to 0.5 microns, from 50 nanometers to 0.4 microns, from 50 nanometers to 0.3 microns, from 50 nanometers to 0.2 microns, from 50 nanometers to 0.1 microns, from 50 nanometers to 0.05 microns, from 50 nanometers to 0.04 microns, or from 50 nanometers to 0.03 microns. In some cases, fibers of this size may be referred to as nanofibers.

In some cases, the topsheet layer has a thickness ranging from 25 microns to 500 microns, e.g., from 25 microns to 400 microns, from 35 microns to 300 microns, or from 50 microns to 275 microns. In terms of upper limits, the topsheet layer may have a thickness less than 500 microns, e.g., less than 400 microns, less than 300 microns, or less than 275 microns. In terms of lower limits, the topsheet layer may have a thickness greater than 25 microns, e.g., greater than 35 microns, greater than 50 microns, or greater than 60 microns.

It has been found that the topsheet layer may advantageously be composed of a relatively hydrophilic and/or hygroscopic material. A polymer of increased hydrophilicity and/or hygroscopy may better attract and hold moisture to which to the AM/AV material is exposed. As discussed below, improved, e.g., increased, hydrophilicity and/or hygroscopy may be accomplished by utilizing the polymer compositions described herein. Thus, it is particularly beneficial to form the topsheet layer, e.g., the first fabric, from a disclosed polymer composition.

### Backsheet Layer

The disclosed AM/AV materials may include a second layer, e.g., a backsheet layer. Generally, the backsheet layer is designed to isolate the absorbent area. For example, when incorporated into the AM/AV material, the backsheet layer may be the outermost layer, farthest from the user's body. In this example, the backsheet layer is the first point of separation from the atmosphere.

Some backsheet layers are well-known in the aforementioned industries. The backsheet layer is composed of a second (outer) fabric. The structure of the outer fabric is not particularly limited. In some embodiments, the outer fabric is a woven fabric. In some embodiments, the outer fabric is a nonwoven fabric. In some embodiments, the backsheet fabric is a knit fabric. In some cases, the backsheet fabric comprises polyamide fibers, e.g., polyamide microfibers or polyamide nanofibers.

The composition of the backsheet layer (e.g., the composition of the outer fabric and/or the fibers thereof) may vary widely. In some embodiments, the outer fabric and/or the fibers thereof are made from and/or comprises the polymer composition, e.g., the polyamide composition, which is discussed in detail below. The polyamide composition comprises a polymer and an AM/AV compound, and in some cases, the AM/AV compound provided for the AM/AV benefits.

In some cases, the outer fabric is conventional polymer fabric. For example, the outer fabric may comprise a fabric made from polyester, nylon, rayon, polyamide 6, polyamide 6,6, polyethylene (PE), polypropylene (PP), polyethylene terephthalate (PET), polyethylene terephthalate glycol (PETG), co-PET, polybutylene terephthalate (PBT) polylactic acid (PLA), and polytrimethylene terephthalate (PTT).

The basis weight of the backsheet layer (e.g., the basis weight of the outer fabric) may vary widely. In one embodiment, the backsheet layer has a basis weight from 2 g/m² to 50 g/m², e.g., from 5 g/m² to 48 g/m², from 5 g/m² to 45 g/m², from 5 g/m² to 42 g/m², from 5 g/m² to 40 g/m², from 5 g/m² to 30 g/m², from 5 g/m² to 25 g/m², from 5 g/m² to 15 g/m², from 2 g/m² to 15 g/m², from 8 g/m² to 50 g/m², from 8 g/m² to 48 g/m², from 8 g/m² to 45 g/m², from 8 g/m² to 42 g/m², from 8 g/m² to 40 g/m², from 8 g/m² to 20 g/m², 10 g/m² to 50 g/m², from 10 g/m² to 48 g/m², from 10 g/m² to 45 g/m², from 10 g/m² to 42 g/m², from 10 g/m² to 40 g/m², from 10 g/m² to 40 g/m², from 10 g/m² to 35 g/m², from 10 g/m² to 30 g/m², from 10 g/m² to 20 g/m², 12 g/m² to 50 g/m², from 12 g/m² to 48 g/m², from 12 g/m² to 45 g/m², from 12 g/m² to 42 g/m², from 12 g/m² to 40 g/m², 14 g/m² to 50 g/m², from 14 g/m² to 48 g/m², from 14 g/m² to 45 g/m², from 14 g/m² to 42 g/m², from 14 g/m² to 40 g/m², from 20 g/m² to 30 g/m², from 15 g/m² to 25 g/m², or from 15 g/m² to 38 g/m². In some instances, these layer(s) may comprise knit fabrics as disclosed above.

In terms of lower limits, the basis weight of the backsheet layer (e.g., of the outer fabric) may be greater than 5 g/m², e.g., greater than 8 g/m², greater than 10 g/m², greater than 12 g/m², greater than 14 g/m², or greater than 15 g/m². In terms of upper limits, the basis weight of the backsheet layer (e.g., of the outer fabric) may be less than 50 g/m², e.g., less than 48 g/m², less than 45 g/m², less than 42 g/m², less than 40 g/m², less than 35 g/m², less than 34 g/m², less than 30 g/m², less than 27 g/m², less than 25 g/m², less than 21 g/m², less than 20 g/m², less than 15 g/m², or less than 12 g/m². In some cases, the basis weight of the backsheet layer (e.g., of the outer fabric) may be about 10 g/m², about 11 g/m², about 12 g/m², about 13 g/m², about 14 g/m², about 15 g/m², about 16 g/m², about 18 g/m², about 19 g/m², about 20 g/m², about 21 g/m², about 22 g/m², about 23 g/m², about 24 g/m², about 25 g/m², about 26 g/m², about 27 g/m², about 28 g/m², about 29 g/m², about 30 g/m², about 31 g/m², about 32 g/m², about 33 g/m², about 34 g/m², about 35 g/m², about 36 g/m², about 37 g/m², about 38 g/m², about 39 g/m², or about 40 g/m², or a basis weight therebetween.

In some cases, the second backsheet layer comprises polyamide and the outer basis weight ranges from 5 gsm to 25 gsm. In some cases, the first topsheet layer comprises polyamide and the face basis weight ranges from 5 gsm to 25 gsm.

In some cases, the backsheet layer has a thickness ranging from 25 microns to 500 microns, e.g., from 25 microns to 400 microns, from 35 microns to 300 microns, or from 50 microns to 275 microns. In terms of upper limits, the backsheet layer may have a thickness less than 500 microns, e.g., less than 400 microns, less than 300 microns, or less than 275 microns. In terms of lower limits, the backsheet layer may have a thickness greater than 25 microns, e.g., greater than 35 microns, greater than 50 microns, or greater than 60 microns.

In some embodiments, the backsheet layer comprises a plurality of fibers and these fibers may have diameters as noted above with respect to the topsheet layer.

In some cases, the outer fabric is a polymer, e.g., polyamide, fabric made from the polymer compositions described herein.

In addition, because the backsheet layer is designed to isolate the abosorbent area, it is desirable that the backsheet layer exhibit AM/AV properties. During use, the backsheet layer may be the layer most exposed to the environment. Furthermore, the backsheet layer may be exposed to microbes and/or viruses, e.g., on surfaces or other objects, before or after use. Thus, it is particularly beneficial to form the backsheet layer, e.g., the outer fabric or a fiber thereof, from an AM/AV polymer compositions as described herein.

### Absorbent Core

The disclosed AM/AV materials include a third layer, e.g., the absorbent core, which may comprise a third fabric. Generally, the third layer is designed to absorb liquid. The absorbent core materials may vary widely, and many absorbent core materials are well known in the industry. For example, cellulose fibers and super absorbent polymers, e.g., polyacrylates.

In some cases, the absorbent core may be is wrapped with a core wrap. The core wrap material may be a fabric similar to those mentioned herein with respect to the aforementioned layers, e.g., the core wrap material may be formed from the AM/AV composition.

### Further Layers

Some embodiments of the AM/AV materials described herein may include additional layers. In some cases, one or more additional layers are added to improve one or performance characteristics of the AM/AV materials, e.g., absorbent efficiency or comfort. In some cases, one or more additional layers are added to improve suitability for a final use. For example, one or more additional layers may be added to provide comfort and/or improved fit to the user of a AM/AV materials.

For example, the AM/AV materials may comprise a pad layer, which may be employed to provide padding and/or separation from other layers or from outer clothing. The pad layer(s) and the fibers/fabrics thereof may have the characteristics as noted above with respect to the topsheet layer, the backsheet layer, and/or the absorbent core. These pad layers may be made from the AM/AV compositions.

In some cases, the further layers may be similar to the aforementioned layers, but may not contain the AM/AV compound.

In some embodiment, the AM/AV materials may comprise a print layers. Print layers may be employed for decorative purposes or to display printing or printed indicia. Print layer materials may vary widely, and many print layer materials are well known in the industry.

In some cases, the AM/AV material may comprise an indicator. The indicator may be used to indicate moisture, expiration, temperature exposure, etc. The indicator may change appearance, when a trigger condition takes place. The mechanism of the indicator may vary widely. Exemplary mechanisms include dye diffusion, color change, chemical reaction (CO₂ or redox), and/or electrochemical. In some embodiments, the indicator may be in the form of a sticker. In some embodiments, the indicator may be in the form of a token, a visual cue, an insignia. This listing is not all inclusive and other indicators are contemplated.

In some cases, the AM/AV material may comprise an acquisition distribution layer (“ADL”). Generally speaking, the ADL is a layer that wicks moisture/slurry from one layer to another, e.g., from the topsheet into the absorbent core. In some embodiments, the ADL acquires moisture from the bottom of the top sheet and distributes that moisture into the absorbent core in the horizontal direction, e.g., the ADL spreads out the moisture laterally onto the absorbent core. The ADL may also disperse moisture in other directions, e.g., vertically. The ADL may, in some cases, be positioned between the topsheet layer and the absorbent core. Other positions for the ADL are also contemplated, however.

In some embodiments, the ADL is made of an ADL fabric. The ADL fabric may be a fabric as described above with respect to the topsheet layer and/or the backsheet layer. The aforementioned characteristics of these layers (see above) may also be applicable to the ADL. For example, the ADL fabric may be an AM/AV fabric made from the AM/AV compositions as described herein. Other known ADL fabrics are also contemplated, with or without AM/AV properties.

In some cases, the AM/AV material may comprise a textured layer. The textured layer may be positioned outwardly of the backsheet layer in some embodiments. Other positions for the textured layer are also contemplated, however. The textured layer may contact the wearer's clothing during wear. In some cases, the textured layer may face the air if a diaper is worn without outer clothing. In other applications, where the AM/AV material is not worn per se (for example when the AM/AV material is a pet pad), the textured layer may be outward of the backsheet layer and may be exposed.

In some embodiments, the textured layer is made of a textured layer fabric. The textured layer fabric may be a fabric as described above with respect to the topsheet layer and/or the backsheet layer. The aforementioned characteristics of these layers (see above) may also be applicable to the textured layer. For example, the textured layer fabric may be an AM/AV fabric made from the AM/AV compositions described herein. Other known textured layer fabrics are also contemplated, with or without AM/AV properties.

### Specific Configurations

The following layered configurations are provided, for exemplary purposes only.

Exemplary diaper configuration: topsheet layer, ADL, pad layer, pad layer, absorbent core, pad layer, print layer, backsheet layer, optional textured layer.

Adult incontinence configuration: topsheet layer, ADL, pad layer, pad layer, absorbent core, backsheet layer.

Feminine hygiene configuration: topsheet layer, ADL, absorbent core, backsheet layer, release sheet

Pet pad configuration: topsheet layer, ADL, absorbent core (with optional odor core component), backsheet layer, and optional other layers that are disclosed herein.

Hospital pad configuration: topsheet layer, ADL, absorbent core, backsheet layer, and optional other layers that are disclosed herein.

### Physical Characteristics

As noted, each layer of the AM/AV material may benefit from increased hydrophilicity and/or hygroscopy. Each of the layers may benefit from increased hydrophilicity and/or hygroscopy, examples include the topsheet layer. In some embodiments, the topsheet layer and/or the pad layer demonstrates relatively high hydrophilicity and/or hygroscopy.

In some cases, the hydrophilicity and/or hygroscopy of a given layer of the AM/AV material may be measured by saturation. In some cases, the hydrophilicity and/or hygroscopy of a given layer of the AM/AV material may be measured by the amount of water it can absorb (as a percentage of total weight). In some embodiments, the layer is capable of absorbing greater than 1.5 wt. % water, based on the total weight of the polymer, e.g., greater than 2.0 wt. %, greater than 3.0%, greater than 5.0 wt. %, greater than 7.0 wt. %, greater than 10.0 wt. %. or greater than 25.0 wt. %. In terms of ranges, the hydrophilic and/or hygroscopic polymer may be capable of absorbing water in an amount ranging from 1.5 wt. % to 50.0 wt. %, e.g., from 1.5 wt. % to 14.0 wt. %, from 1.5 wt. % to 9.0 wt. %, from 2.0 wt. % to 8 wt. %, from 2.0 wt. % to 7 w%, from 2.5 wt. % to 7 wt. %, or from 1.5 wt. % to 25.0 wt. %,.

In some cases, the hydrophilicity and/or the hygroscopy of a given layer of the AM/AV material may be measured by the water contact angle of the layer. The water contact angle is the angle formed by the interface of a surface of the layer, e.g., of the topsheet layer. Preferably, the contact angle of the layer is measured while the layer is flat (e.g., substantially flat).

In some embodiments, a layer of the AM/AV material demonstrates a water contact angle less than 90° , e.g., less than 85° , less than 80° , or less than 75° . In terms of lower limits, the water contact angle of a layer may be greater than 10° , e.g., greater than 20° , greater than 30° , or greater than 40° . In terms of ranges, the water contact angle of a layer may be from 10° to 90° , e.g., from 10° to 85° , from 10° to 80° , from 10° to 75° , from 20° to 90° , from 20° to 85° , from 20° to 80° , from 20° to 75° , from 30° to 90° , from 30° to 85° , from 30° to 80° , from 30° to 75° , from 40° to 90° , from 40° to 85° , from 40° to 80° , or from 40° to 75° .

As noted, the increased hydrophilicity and/or hygroscopy of AM/AV material may be the result of a polymer composition from which the layer is formed. The polymer compositions described herein, for example, demonstrate increased hydrophilicity and/or hygroscopy and are therefore particularly suitable for the disclosed AM/AV material.

In some embodiments, a polymer may be specially prepared to impart increased hydrophilicity and/or hygroscopy. For example, an increase in hygroscopy may be achieved in the selection and/or modification the polymer. In some embodiments, the polymer may be a common polymer, e.g., a common polyamide, which has been modified to increase hygroscopy. In these embodiments, a functional endgroup modification on the polymer may increase hygroscopy. For example, the polymer may be PA6,6, which has been modified to include a functional endgroup that increases hygroscopy.

### Performance Characteristics

The performance of the AM/AV material described herein may be assessed using a variety of conventional metrics.

Antiodor performance may be measured by toilet odor reduction, as measured in accordance with ISO 17299-3 (2014). In some embodiments, the AM/AV material demonstrates a toilet odor reduction greater than 50% e.g., greater than 60%, greater than 70%, greater than 80%, or greater than 90%. Toilet odor may be tested using specific test chemicals, e.g., ammonia, acetic acid, isovaleric acid, hydrogen sulfide, indole, and/or nonenal. At least one of the layers (or the fibers thereof) demonstrates the toilet odor reduction for one or more of these test chemicals.

In some cases, the AM/AV performance relates to antifungal performance. The antifungal activity of the AM/AV materials may be measured by the standard procedure defined by Mod. E3160. In one embodiment, the AM/AV materials inhibits the growth (growth reduction) of Candida auris or Candida albicans in an amount greater than 10% fungal growth, e.g., greater than 20%, greater than 30%, greater than 40%, greater than 50%, greater than 60%, greater than 70%, greater than 80%, greater than 90% or greater than 93%.

In some cases, the performance relates to comfort performance. The comfort of the AM/AV materials may be measured by the standard procedure defined by cup crush testing, as defined be NWSP 402.0. Another measurement method is the use of the FTT Fabric Touch Tester from SDL Atlas.

Rewet testing is commonly known in the industry. Generally speaking, rewet testing determines the capacity of a topsheet/coverstock to hold back liquids even under the pressure of weight, e.g., how much wetness returns to the skin during use. Test methods and equipment are well known. In some embodiments, the AM/AV material (and/or the fibers of the various layers) may demonstrate a rewet value less than 5 g after a first water application, e.g., less than 4 g, less than 3 g, less than 2 g, less than 1 g, less than 0.8 g, less than 0.7 g, less than 0.5 g, or less than 0.3 g. In some cases, these rewet values are suitable for use (while also providing the aforementioned AM/AV benefits). In some embodiments, the AM/AV material (and/or the fibers of the various layers) may demonstrate a rewet value less than 35 g after a fourth water application, e.g., less than 30 g, less than 25 g, less than 20 g, less than 15 g, less than 13 g, less than 12 g, less than 10 g, less than 9.5 g, or less than 9 g. In some cases, these rewet values are suitable for use (while also providing the aforementioned AM/AV benefits).

Strikethrough testing is commonly known in the industry. Generally speaking, strikethrough testing determines the ability of topsheet/coverstock to resist the transport back onto the skin of a liquid that has already penetrated the coverstock. One test method is NWSP 70.8. Testing equipment is well known and commercially available. A strikethrough test is disclosed in EP 1187588 B1. In some embodiments, the AM/AV material (and/or the fibers of the various layers) may demonstrate a strikethrough value less than 25 seconds after a first water application, e.g., less than 22 seconds, less than 20 second, less than 18 seconds, or less than 16 seconds. In some embodiments, the AM/AV material (and/or the fibers of the various layers) may demonstrate a strikethrough value less than 40 seconds after a fourth water application, e.g., less than 35 seconds, less than 32 second, less than 30 seconds, less than 28 seconds, or less than 25 seconds.

Bacterial filtration efficiency (or “BFE”) measures how well the AM/AV material traps or isolates bacteria when exposed to a bacteria-containing aerosol. BFE measures a percentage of bacteria that trapped or isolated by the AM/AV material. ASTM International specifies testing with a droplet size of 3.0 microns containing Staph. aureus (average size 0.6-0.8 microns).

In some embodiments, the AM/AV material demonstrates a BFE greater than 90%, e.g., greater than 92%, greater than 93%, greater than 94%, greater than 95%, greater than 97%, greater than 98%, greater than 99%, greater than 99.5%, greater than 99.9%, or greater than 99.99%. In terms upper limits, the AM/AV material may demonstrate a BFE less than 100%, e.g., less than 99.999%, less than 99.995%, less than 99.99%, or less than 99.95%.

In some embodiments, the AM/AV material demonstrates a BFE of about 90%, about 91%, about 92%, about 93%, about 94%, about 95%, about 95.5%, about 96%, about 96.5%, about 97%, about 97.5%, about 98%, about 98.5%, about 99%, about 99.2%, about 99.3%, about 99.4%, about 99.5%, about 99.8%, about 99.9%, about 99.95%, or about 99.99%, or any percentage therebetween.

As has been noted, in some embodiments, the AM/AV materials may demonstrate AM/AV activity. In some cases, the AM/AV activity may be the result of the polymer composition from which the AM/AV materials or the layers/fabrics thereof or the fibers thereof are formed. For example, the AM/AV activity may be the result of forming the AM/AV materials from a polymer composition described herein.

In some embodiments, at least one of the layers demonstrates AM/AV activity. In some embodiments, a combination of the layers demonstrates AM/AV activity. In some embodiments, the entire AM/AV materials demonstrates AM/AV properties.

In some embodiments, the AM/AV materials exhibit permanent, e.g., near permanent, AM/AV properties. Said another way, the AM/AV properties of the polymer composition last for a prolonged period of time, e.g., longer than one or more day, longer than one or more week, longer than one or more month, or longer than one or more years.

The AM/AV properties may include any antimicrobial effect. In some embodiments, for example, the antimicrobial properties of the AM/AV material include limiting, reducing, or inhibiting infection of a microbe, e.g., a bacterium or bacteria. In some embodiments, the antimicrobial properties of the AM/AV material include limiting, reducing, or inhibiting growth and/or killing a bacterium. In some cases, the AM/AV material may limit, reduce, or inhibit both infection and growth of a bacterium.

The bacterium or bacteria affected by the antimicrobial properties of the AM/AV material are not particularly limited. In some embodiments, for example, the bacterium is a Streptococcus bacterium (e.g., Streptococcus pneumonia, Streptococcus pyogenes), a Staphylococcus bacterium (e.g., Staphylococcus aureus, Methicillin-resistant Staphylococcus aureus (MRSA)), a Peptostreptococcus bacterium (e.g., Peptostreptococcus anaerobius, Peptostreptococcus asaccharolyticus), a coli bacterium (e.g., Escherichia coli), or a Mycobacterium bacterium, (e.g., Mycobacterium tuberculosis), a Mycoplasma bacterium (e.g., Mycoplasma adleri, Mycoplasma agalactiae, Mycoplasma agassizii, Mycoplasma amphoriforme, Mycoplasma fermentans, Mycoplasma genitalium, Mycoplasma haemofelis, Mycoplasma hominis, Mycoplasma hyopneumoniae, Mycoplasma hyorhinis, Mycoplasma pneumoniae). In some embodiments, the antimicrobial properties include limiting, reducing, or inhibiting the infection or pathogenesis of multiple bacteria, e.g., a combination of two or more bacteria from the above list.

The antimicrobial activity of the AM/AV materials may be measured by the standard procedure defined by ISO 20743:2013. This procedure measures antimicrobial activity by determining the percentage of a given bacterium or bacteria, e.g. Staphylococcus aureus, inhibited by a tested fiber. In one embodiment, the AM/AV material inhibits the growth (growth reduction) of S. aureus in an amount ranging from 60% to 100%, e.g., from 60% to 99.999999%, from 60% to 99.99999%, from 60% to 99.9999%, from 60% to 99.999% from 60% to 99.999%, from 60% to 99.99%, from 60% to 99.9%, from 60% to 99%, from 60% to 98%, from 60% to 95%, from 65% to 99.999999%, from 65% to 99.99999%, from 65% to 99.9999%, from 65% to 99.999% from 65% to 99.999%, from 65% to 100%, from 65% to 99.99%, from 65% to 99.9%, from 65% to 99%, from 65% to 98%, from 65% to 95%, from 70% to 100%, from 70% to 99.999999%, from 70% to 99.99999%, from 70% to 99.9999%, from 70% to 99.999% from 70% to 99.999%, from 70% to 99.99%, from 70% to 99.9%, from 70% to 99%, from 70% to 98%, from 70% to 95%, from 75% to 100%, from 75% to 99.99%, from 75% to 99.9%, from 75% to 99.999999%, from 75% to 99.99999%, from 75% to 99.9999%, from 75% to 99.999% from 75% to 99.999%, from 75% to 99%, from 75% to 98%, from 75% to 95%, %, from 80% to 99.999999%, from 80% to 99.99999%, from 80% to 99.9999%, from 80% to 99.999% from 80% to 99.999%, from 80% to 100%, from 80% to 99.99%, from 80% to 99.9%, from 80% to 99%, from 80% to 98%, or from 80% to 95%. In terms of lower limits, the AM/AV material may inhibit greater than 60% growth of S. aureus, e.g., greater than 65%, greater than 70%, greater than 75%, greater than 80%, greater than 85%, greater than 90%, greater than 95%, greater than 98%, greater than 99%, greater than 99.9%, greater than 99.99%, greater than 99.999%, greater than 99.9999%, greater than 99.99999%, or greater than 99.999999%.

Klebsiella pneumoniae efficacy may also be determined using the aforementioned tests. In some embodiments, a product formed from the polymer composition inhibits the growth (growth reduction) of Klebsiella pneumoniae, as measured by the test mentioned above. Escherichia coli may be determined using ASTM E3160 (2018). The ranges and limits for Staph aureus are applicable to Escherichia coli and/or Klebsiella pneumoniae and/or SARS-CoV-2 as well.

Efficacy may be characterized in terms of log reduction. In terms of Escherichia coli log reduction, the composition/fibers/fabrics may be determined via ASTM 3160 (2018) and may demonstrate a coli log reduction greater than 1.5, e.g., greater than 2.0, greater than 2.15, greater than 2.5, greater than 2.7, greater than 3.0, greater than 3.3, greater than 4.0, greater than 4.1, greater than 5.0, or greater than 6.0.

In terms of Staph aureus log reduction, the composition/fibers/fabrics may be determined via ISO 20743:2013 and may demonstrate a microbial log reduction greater than 1.5, e.g., greater than 2.0, greater than 2.5, greater than 2.6, greater than 2.7, greater than 3.0, greater than 4.0, greater than 5.0, greater than 6.0, or greater than 7.0.

In terms of Klebsiella pneumoniae log reduction, the composition/fibers/fabrics may be determined via ISO 20743:2013 and may demonstrate a microbial log reduction greater than 1.5, e.g., greater than 2.0, greater than 2.4, greater than 2.5, greater than 2.6, greater than 3.0, greater than 4.0, greater than 5.0, greater than 6.0, greater than 7.0, or greater than 8.0.

In terms of SARS-CoV-2 log reduction, the composition/fibers/fabrics may be determined via ISO 18184:2019 and may demonstrate a viral log reduction greater than 1.5, e.g., greater than 2.0, greater than 2.5, greater than 2.6, greater than 1.7, greater than 3.0, greater than 4.0, greater than 5.0, or greater than 6.0.

The AM/AV properties may include any antiviral effect. In some embodiments, for example, the antiviral properties of the AM/AV material include limiting, reducing, or inhibiting infection of a virus. In some embodiments, the antiviral properties of the AM/AV material include limiting, reducing, or inhibiting pathogenesis of a virus. In some cases, the polymer composition may limit, reduce, or inhibit both infection and pathogenesis of a virus.

The virus affected by the antiviral properties of the AM/AV material is not particularly limited. In some embodiments, for example, the virus is an adenovirus, a herpesvirus, an ebolavirus, a poxvirus, a rhinovirus, a coxsackievirus, an arterivirus, an enterovirus, a morbillivirus, a coronavirus, an influenza A virus, an avian influenza virus, a swine-origin influenza virus, or an equine influence virus. In some embodiments, the antiviral properties include limiting, reducing, or inhibiting the infection or pathogenesis of one of virus, e.g., a virus from the above list. In some embodiments, the antiviral properties include limiting, reducing, or inhibiting the infection or pathogenesis of multiple viruses, e.g., a combination of two or more viruses from the above list.

In some cases, the virus is a coronavirus, e.g., severe acute respiratory syndrome coronavirus (SARS-CoV), Middle East respiratory syndrome coronavirus (MERS-CoV), or severe acute respiratory syndrome coronavirus 2 (SARS-CoV-2) (e.g., the coronavirus that causes COVID-19). In some cases, the virus is structurally related to a coronavirus.

In some cases, the virus is an influenza virus, such as an influenza A virus, an influenza B virus, an influenza C virus, or an influenza D virus, or a structurally related virus. In some cases, the virus is identified by an influenza A virus subtype, e.g., H1N1, H1N2, H2N2, H2N3, H3N1, H3N2, H3N8, H5N1, H5N2, H5N3, H5N6, H5N8, H5N9, H6N1, H7N1, H7N4, H7N7, H7N9, H9N2, or H10N7.

In some cases, the virus is a bacteriophage, such as a linear or circular single-stranded DNA virus (e.g., phi X 174 (sometimes referred to as ΦTX174)), a linear or circular double-stranded DNA, a linear or circular single-stranded RNA, or a linear or circular double-stranded RNA. In some cases, the antiviral properties of the polymer composition may be measured by testing using a bacteriophage, e.g., phi X 174.

In some cases, the virus is an ebolavirus, e.g., Bundibugyo ebolavirus (BDBV), Reston ebolavirus (RESTV), Sudan ebolavirus (SUDV), Taï Forest ebolavirus (TAFV), or Zaire ebolavirus (EBOV). In some cases, the virus is structurally related to an ebolavirus.

The antiviral activity may be measured by a variety of conventional methods. For example, ISO 18184:2019 may be utilized to assess the antiviral activity. In one embodiment, the AM/AV material inhibits the pathogenesis (e.g., growth) of a virus in an amount ranging from 60% to 100%, e.g., from 60% to 99.999999%, from 60% to 99.99999%, from 60% to 99.9999%, from 60% to 99.999% from 60% to 99.999%, from 60% to 99.99%, from 60% to 99.9%, from 60% to 99%, from 60% to 98%, from 60% to 95%, from 65% to 99.999999%, from 65% to 99.99999%, from 65% to 99.9999%, from 65% to 99.999% from 65% to 99.999%, from 65% to 100%, from 65% to 99.99%, from 65% to 99.9%, from 65% to 99%, from 65% to 98%, from 65% to 95%, from 70% to 100%, from 70% to 99.999999%, from 70% to 99.99999%, from 70% to 99.9999%, from 70% to 99.999% from 70% to 99.999%, from 70% to 99.99%, from 70% to 99.9%, from 70% to 99%, from 70% to 98%, from 70% to 95%, from 75% to 100%, from 75% to 99.99%, from 75% to 99.9%, from 75% to 99.999999%, from 75% to 99.99999%, from 75% to 99.9999%, from 75% to 99.999% from 75% to 99.999%, from 75% to 99%, from 75% to 98%, from 75% to 95%, %, from 80% to 99.999999%, from 80% to 99.99999%, from 80% to 99.9999%, from 80% to 99.999% from 80% to 99.999%, from 80% to 100%, from 80% to 99.99%, from 80% to 99.9%, from 80% to 99%, from 80% to 98%, or from 80% to 95%. In terms of lower limits, a AM/AV material may inhibit greater than 60% of pathogenesis of the virus, e.g., greater than 65%, greater than 70%, greater than 75%, greater than 80%, greater than 85%, greater than 90%, greater than 95%, greater than 98%, greater than 99%, greater than 99.9%, greater than 99.99%, greater than 99.999%, greater than 99.9999%, greater than 99.99999%, or greater than 99.999999%.

In addition, the use of the polymer compositions disclosed herein provides for biocompatibility advantages. For example, the overall softness of the aforementioned fabrics, along with the compositional characteristics, provides for unexpected reductions in irritation and sensitivity. Beneficially, the disclosed fibers and fabric do not demonstrate the biocompatibility issues associated with conventional fabrics, e.g., those that employ metals with toxicity problems such as silver. For example, the various AM/AV mask configurations demonstrate passing results with regard to irritation and sensitization, as tested in accordance with ISO 10993-10 and 10993-12.

### AM/AV Polymer Composition

As noted above, the AM/AV materials of the present disclosure may comprise polymer compositions that beneficially exhibit antimicrobial and/or antiviral properties. For example, the topsheet layer and/or the pad layer, may be made from and/or may comprise an antimicrobial/antiviral polymer composition as described herein.

Polymer compositions suitable for use in the AM/AV materials described herein generally comprise a polymer and one or more AM/AV compounds, e.g., metals (e.g., metallic compounds). In some embodiments, the polymer compositions comprise a polymer, zinc (provided to the composition via a zinc compound), and/or phosphorus (provided to the composition via a phosphorus compound). In some embodiments, the polymer compositions comprise a polymer, copper (provided to the composition via a copper compound), and phosphorus (provided to the composition via a phosphorus compound).

Exemplary polymer compositions are disclosed in U.S. patent application Ser. No. 17/192,491, filed Mar. 4, 2021, and U.S. patent application Ser. No. 17/192,533, filed on Mar. 4, 2021, both of which are incorporated herein by reference.

### Polymer

The polymer compositions comprise a polymer, which, in some embodiments, is a polymer suitable for producing fibers and fabrics. In one embodiment, the polymer composition comprises a polymer in an amount ranging from 50 wt. % to 100 wt. %, e.g., from 50 wt. % to 99.99 wt. %, from 50 wt. % to 99.9 wt. %, from 50 wt. % to 99 wt. % from 55 wt. % to 100 wt. %, from 55 wt. % to 99.99 wt. %, from 55 wt. % to 99.9 wt. %, from 55 wt. % to 99 wt. %, from 60 wt. % to 100 wt. %, from 60 wt. % to 99.99 wt. %, from 60 wt. % to 99.9 wt. %, from 60 wt. % to 99 wt. %., from 65 wt. % to 100 wt. %, from 65 wt. % to 99.99 wt. %, from 65 wt. % to 99.9 wt. %, or from 65 wt. % to 99 wt. %. In terms of upper limits, the polymer composition may comprise less than 100 wt. % of the polymer, e.g., less than 99.99 wt. %, less than 99.9 wt. %, or less than 99 wt. %. In terms of lower limits, the polymer composition may comprise greater than 50 wt. % of the polymer, e.g., greater than 55 wt. %, greater than 60 wt. %, or greater than 65 wt. %.

The polymer of the polymer composition may vary widely. The polymer may include but is not limited to, a thermoplastic polymer, polyester, nylon, rayon, polyamide 6, polyamide 6,6, polyethylene (PE), polypropylene (PP), polyethylene terephthalate (PET), polyethylene terephthalate glycol (PETG), co-PET, polybutylene terephthalate (PBT) polylactic acid (PLA), and polytrimethylene terephthalate (PTT). In some embodiments, the polymer composition may comprise PET, for its strength, longevity during washing, ability to be made permanent press, and ability to be blended with other fibers. In some embodiments, the polymer may be PA6,6. In some cases, nylon is known to be a stronger fiber than PET and exhibits a non-drip burning characteristic that is beneficial, e.g., in military or automotive textile applications, and is more hydrophilic than PET. The polymer used in the present disclosure can be a polyamide, polyether amide, polyether ester or polyether urethane or a mixture thereof.

In some cases, the polymer compositions may comprise polyethylene. Suitable examples of polyethylene include linear low-density polyethylene (LLDPE), low-density polyethylene (LDPE), medium-density polyethylene (MDPE), high-density polyethylene (HDPE), and ultra-high-molecular-weight polyethylene (UHMWPE).

In some cases, the polymer compositions may comprise polycarbonate (PC). For example, the polymer composition may comprise a blend of polycarbonate with other polymers, e.g., a blend of polycarbonate and acrylonitrile butadiene styrene (PC-ABS), a blend of polycarbonate and polyvinyl toluene (PC-PVT), a blend of polycarbonate and polybutylene terephthalate (PC-PBT), a blend of polycarbonate and polyethylene terephthalate (PC-PET), or combinations thereof.

In some cases, the polymer composition may comprise polyamides. Common polyamides include nylons and aramids. For example, the polyamide may comprise PA-4T/4I; PA-4T/6I; PA-5T/5I; PA-6; PA6,6; PA6,6/6; PA6,6/6T; PA-6T/6I; PA-6T/6I/6; PA-6T/6; PA-6T/6I/66; PA-6T/MPMDT (where MPMDT is polyamide based on a mixture of hexamethylene diamine and 2-methylpentamethylene diamine as the diamine component and terephthalic acid as the diacid component); PA-6T/66; PA-6T/610; PA-10T/612; PA-10T/106; PA-6T/612; PA-6T/10T; PA-6T/10I; PA-9T; PA-10T; PA-12T; PA-10T/10I; PA-10T/12; PA-10T/11; PA-6T/9T; PA-6T/12T; PA-6T/10T/6I; PA-6T/6I/6; PA-6T/61/12; and copolymers, blends, mixtures and/or other combinations thereof. Additional suitable polyamides, additives, and other components are disclosed in U.S. patent application Ser. No. 16/003,528.

In some embodiments, the polymer compositions comprise a thermoplastic polymer, polyester, nylon, rayon, polyamide, polyamide, poly olefin, polyolefin terephthalate, polyolefin terephthalate glycol, co-PET, or polylactic acid, or combinations thereof.

The polymer composition may, in some embodiments, comprise a combination of polyamides. By combining various polyamides, the final composition may be able to incorporate the desirable properties, e.g., mechanical properties, of each constituent polyamides. For example, in some embodiments, the polyamide comprises a combination of PA-6, PA6,6, and PA6,6/6T. In these embodiments, the polyamide may comprise from 1 wt. % to 99 wt. % PA-6, from 30 wt. % to 99 wt. % PA6,6, and from 1 wt. % to 99 wt. % PA6,6/6T. In some embodiments, the polyamide comprises one or more of PA-6, PA6,6, and PA6,6/6T. In some aspects, the polymer composition comprises 6 wt. % of PA-6 and 94 wt. % of PA6,6. In some aspects, the polymer composition comprises copolymers or blends of any of the polyamides mentioned herein.

The polymer composition may also comprise polyamides produced through the ring-opening polymerization or polycondensation, including the copolymerization and/or copolycondensation, of lactams. Without being bound by theory, these polyamides may include, for example, those produced from propriolactam, butyrolactam, valerolactam, and caprolactam. For example, in some embodiments, the polyamide is a polymer derived from the polymerization of caprolactam. In those embodiments, the polymer comprises at least 10 wt. % caprolactam, e.g., at least 15 wt. %, at least 20 wt. %, at least 25 wt. %, at least 30 wt. %, at least 35 wt. %, at least 40 wt. %, at least 45 wt. %, at least 50 wt. %, at least 55 wt. %, or at least 60 wt. %. In some embodiments, the polymer includes from 10 wt. % to 60 wt. % of caprolactam, e.g., from 15 wt. % to 55 wt. %, from 20 wt. % to 50 wt. %, from 25 wt. % to 45 wt. %, or from 30 wt. % to 40 wt. %. In some embodiments, the polymer comprises less than 60 wt. % caprolactam, e.g., less than 55 wt. %, less than 50 wt. %, less than 45 wt. %, less than 40 wt. %, less than 35 wt. %, less than 30 wt. %, less than 25 wt. %, less than 20 wt. %, or less than 15 wt. %. Furthermore, the polymer composition may comprise the polyamides produced through the copolymerization of a lactam with a nylon, for example, the product of the copolymerization of a caprolactam with PA6,6.

In some aspects, the polymer can formed by conventional polymerization of the polymer composition in which an aqueous solution of at least one diamine-carboxylic acid salt is heated to remove water and effect polymerization to form an antiviral nylon. This aqueous solution is preferably a mixture which includes at least one polyamide-forming salt in combination with the specific amounts of a zinc compound, a copper compound, and/or a phosphorus compound described herein to produce a polymer composition. Conventional polyamide salts are formed by reaction of diamines with dicarboxylic acids with the resulting salt providing the monomer. In some embodiments, a preferred polyamide-forming salt is hexamethylenediamine adipate (nylon 6,6 salt) formed by the reaction of equimolar amounts of hexamethylenediamine and adipic acid.

### AM/AV (Metallic) Compounds

As noted above, the polymer composition may include one or more AM/AV compounds, which may be in the form of a metallic compound. In some embodiments, the polymer composition includes zinc, e.g., in a zinc compound, optionally phosphorus, e.g., in a phosphorus compound, optionally copper, e.g., in a copper compound, optionally silver, e.g., in a silver compound, or combinations thereof. As used herein, a metallic compound refers to a compound having at least one metal molecule or ion, e.g., a “zinc compound” refers to a compound having at least one zinc molecule or ion.

Some conventional polymer compositions, fibers and fabrics utilize AM/AV compounds to inhibit viruses and other pathogens. For example, some fabrics may include antimicrobial additives, e.g., silver, coated or applied as a film on an exterior surface. However, it has been found that these treatments or coatings often present a host of problems. For example, the coated additives may extract out of the fibers/fabric during dyeing or washing processes, which adversely affects the antimicrobial and/or antiviral properties. As it relates to conventional products, while in constant use, some coatings, e.g., silver, may contribute to health and/or even environmental problems. In contrast to conventional formulations, the polymer compositions disclosed herein comprise a unique combination of AM/AV compounds (e.g., metallic compounds) rather than simply coating the AM/AV compounds on a surface. Stated another way, the polymer composition may have certain amounts of a metallic compound embedded in the polymer matrix such that the polymer composition retains AM/AV properties during and after dyeing and/or washing.

In one embodiment, AM/AV compounds can be added as a masterbatch. The masterbatch may include a polyamide such as nylon 6 or nylon 6,6. Other masterbatch compositions are contemplated.

The polymer composition may comprise metallic compounds, e.g., a metal or a metallic compound, dispersed within the polymer composition. In one embodiment, the polymer composition comprises metallic compounds in an amount ranging from 5 wppm to 20,000 wppm, e.g., from 5 wppm to 17,500 wppm, from 5 wppm to 17,000 wppm, from 5 wppm to 16,500 wppm, from 5 wppm to 16,000 wppm, from 5 wppm to 15,500 wppm, from 5 wppm to 15,000 wppm, from 5 wppm to 12,500 wppm, from 5 wppm to 10,000 wppm, from 5 wppm to 5000 wppm, from 5 wppm to 4000 wppm, e.g., from 5 wppm to 3000 wppm, from 5 wppm to 2000 wppm, from 5 wppm to 1000 wppm, from 5 wppm to 500 wppm, from 10 wppm to 20,000 wppm, from 10 wppm to 17,500 wppm, from 10 wppm to 17,000 wppm, from 10 wppm to 16,500 wppm, from 10 wppm to 16,000 wppm, from 10 wppm to 15,500 wppm, from 10 wppm to 15,000 wppm, from 10 wppm to 12,500 wppm, from 10 wppm to 10,000 wppm, from 10 wppm to 5000 wppm, from 10 wppm to 4000 wppm, from 10 wppm to 3000 wppm, from 10 wppm to 2000 wppm, from 10 wppm to 1000 wppm, from 10 wppm to 500 wppm, from 50 wppm to 20,000 wppm, from 50 wppm to 17,500 wppm, from 50 wppm to 17,000 wppm, from 50 wppm to 16,500 wppm, from 50 wppm to 16,000 wppm, from 50 wppm to 15,500 wppm, from 50 wppm to 15,000 wppm, from 50 wppm to 12,500 wppm, from 50 wppm to 10,000 wppm, from 50 wppm to 5000 wppm, from 50 wppm to 4000 wppm, from 50 wppm to 3000 wppm, from 50 wppm to 2000 wppm, from 50 wppm to 1000 wppm, from 50 wppm to 500 wppm, from 100 wppm to 20,000 wppm, from 100 wppm to 17,500 wppm, from 100 wppm to 17,000 wppm, from 100 wppm to 16,500 wppm, from 100 wppm to 16,000 wppm, from 100 wppm to 15,500 wppm, from 100 wppm to 15,000 wppm, from 100 wppm to 12,500 wppm, from 100 wppm to 10,000 wppm, from 100 wppm to 5000 wppm, from 100 wppm to 4000 wppm, from 100 wppm to 3000 wppm, from 100 wppm to 2000 wppm, from 100 wppm to 1000 wppm, from 100 wppm to 500 wppm, from 200 wppm to 20,000 wppm, from 200 wppm to 17,500 wppm, from 200 wppm to 17,000 wppm, from 200 wppm to 16,500 wppm, from 200 wppm to 16,000 wppm, from 200 wppm to 15,500 wppm, from 200 wppm to 15,000 wppm, from 200 wppm to 12,500 wppm, from 200 wppm to 10,000 wppm, from 200 wppm to 5000 wppm, from 200 wppm to 4000 wppm, from 200 wppm to 3000 wppm, from 200 wppm to 2000 wppm, from 200 wppm to 1000 wppm, from 200 wppm to 900 wppm, from 400 wppm to 700 wppm, from 475 wppm to 625 wppm, from 500 wppm to 600 wppm, or from 200 wppm to 500 wppm.

In terms of lower limits, the polymer composition may comprise greater than 5 wppm metallic compounds, e.g., greater than 10 wppm, greater than 50 wppm, greater than 100 wppm, greater than 200 wppm, greater than 300 wppm greater than 400 wppm, greater than 475 wppm, greater than 500 wppm. In terms of upper limits, the polymer composition may comprise less than 20,000 wppm metallic compounds, e.g., less than 17,500 wppm, less than 17,000 wppm, less than 16,500 wppm, less than 16,000 wppm, less than 15,500 wppm, less than 15,000 wppm, less than 12,500 wppm, less than 10,000 wppm, less than 5000 wppm, less than less than 4000 wppm, less than 3000 wppm, less than 2000 wppm, less than 1000 wppm, less than 900 wppm, less than 700 wppm, less than 625 wppm, less than 600 wppm, or less than 500 wppm. As noted above, the metallic compounds are preferably embedded in the polymer formed from the polymer composition.

As noted above, the polymer composition includes zinc in a zinc compound and phosphorus in a phosphorus compound, preferably in specific amounts in the polymer composition, to provide the aforementioned structural and antiviral benefits. As used herein, “zinc compound” refers to a compound having at least one zinc molecule or ion (likewise for copper compounds). As used herein, “phosphorus compound” refers to a compound having at least one phosphorus molecule or ion. Zinc content may be indicated by zinc or zinc ion (the same is true for copper). The ranges and limits may be employed for zinc content and for zinc ion content, and for other metal content, e.g., copper content. The calculation of zinc ion content based on zinc or zinc compound can be made by the skilled chemist, and such calculations and adjustments are contemplated.

The inventors have found that the use of specific zinc compounds (and the zinc contained therein) and phosphorus compounds (and the phosphorus contained therein) at specific molar ratios minimizes the negative effects of the zinc compound on the polymer composition. For example, too much zinc compound in the polymer composition can lead to decreased polymer viscosity and inefficiencies in production processes.

The polymer composition may comprise zinc, e.g., in a zinc compound or as zinc ion, e.g., zinc or a zinc compound, dispersed within the polymer composition. In one embodiment, the polymer composition comprises zinc in an amount ranging from 5 wppm to 20,000 wppm, e.g., from 5 wppm to 17,500 wppm, from 5 wppm to 17,000 wppm, from 5 wppm to 16,500 wppm, from 5 wppm to 16,000 wppm, from 5 wppm to 15,500 wppm, from 5 wppm to 15,000 wppm, from 5 wppm to 12,500 wppm, from 5 wppm to 10,000 wppm, from 5 wppm to 5000 wppm, from 5 wppm to 4000 wppm, e.g., from 5 wppm to 3000 wppm, from 5 wppm to 2000 wppm, from 5 wppm to 1000 wppm, from 5 wppm to 500 wppm, from 10 wppm to 20,000 wppm, from 10 wppm to 17,500 wppm, from 10 wppm to 17,000 wppm, from 10 wppm to 16,500 wppm, from 10 wppm to 16,000 wppm, from 10 wppm to 15,500 wppm, from 10 wppm to 15,000 wppm, from 10 wppm to 12,500 wppm, from 10 wppm to 10,000 wppm, from 10 wppm to 5000 wppm, from 10 wppm to 4000 wppm, from 10 wppm to 3000 wppm, from 10 wppm to 2000 wppm, from 10 wppm to 1000 wppm, from 10 wppm to 500 wppm, from 50 wppm to 20,000 wppm, from 50 wppm to 17,500 wppm, from 50 wppm to 17,000 wppm, from 50 wppm to 16,500 wppm, from 50 wppm to 16,000 wppm, from 50 wppm to 15,500 wppm, from 50 wppm to 15,000 wppm, from 50 wppm to 12,500 wppm, from 50 wppm to 10,000 wppm, from 50 wppm to 5000 wppm, from 50 wppm to 4000 wppm, from 50 wppm to 3000 wppm, from 50 wppm to 2000 wppm, from 50 wppm to 1000 wppm, from 50 wppm to 500 wppm, from 100 wppm to 20,000 wppm, from 100 wppm to 17,500 wppm, from 100 wppm to 17,000 wppm, from 100 wppm to 16,500 wppm, from 100 wppm to 16,000 wppm, from 100 wppm to 15,500 wppm, from 100 wppm to 15,000 wppm, from 100 wppm to 12,500 wppm, from 100 wppm to 10,000 wppm, from 100 wppm to 5000 wppm, from 100 wppm to 4000 wppm, from 100 wppm to 3000 wppm, from 100 wppm to 2000 wppm, from 100 wppm to 1000 wppm, from 100 wppm to 500 wppm, from 200 wppm to 20,000 wppm, from 200 wppm to 17,500 wppm, from 200 wppm to 17,000 wppm, from 200 wppm to 16,500 wppm, from 200 wppm to 16,000 wppm, from 200 wppm to 15,500 wppm, from 200 wppm to 15,000 wppm, from 200 wppm to 12,500 wppm, from 200 wppm to 10,000 wppm, from 200 wppm to 5000 wppm, from 200 wppm to 4000 wppm, 5000 wppm to 20000 wppm, from 200 wppm to 3000 wppm, from 200 wppm to 2000 wppm, from 200 wppm to 1000 wppm, from 200 wppm to 500 wppm, from 10 wppm to 900 wppm, from 200 wppm to 900 wppm, from 425 wppm to 600 wppm, from 425 wppm to 525 wppm, from 350 wppm to 600 wppm, from 375 wppm to 600 wppm, from 375 wppm to 525 wppm, from 480 wppm to 600 wppm, from 480 wppm to 525 wppm, from 600 wppm to 750 wppm, or from 600 wppm to 700 wppm.

In terms of lower limits, the polymer composition may comprise greater than 5 wppm of zinc, e.g., greater than 10 wppm, greater than 50 wppm, greater than 100 wppm, greater than 200 wppm, greater than 300 wppm, greater than 350 wppm, greater than 375 wppm, greater than 400 wppm, greater than 425 wppm, greater than 480 wppm, greater than 500 wppm, or greater than 600 wppm.

In terms of upper limits, the polymer composition may comprise less than 20,000 wppm of zinc, e.g., less than 17,500 wppm, less than 17,000 wppm, less than 16,500 wppm, less than 16,000 wppm, less than 15,500 wppm, less than 15,000 wppm, less than 12,500 wppm, less than 10,000 wppm, less than 5000 wppm, less than less than 4000 wppm, less than 3000 wppm, less than 2000 wppm, less than 1000 wppm, less than 500 wppm, less than 400 wppm, less than 330 wppm, less than 300. In some aspects, the zinc compound is embedded in the polymer formed from the polymer composition.

The ranges and limits are applicable to both zinc in elemental or ionic form and to zinc compound. The same is true for other ranges and limits disclosed herein relating to other metals, e.g., copper. For example, the ranges may relate to the amount of zinc ions dispersed in the polymer.

The zinc of the polymer composition is present in or provided via a zinc compound, which may vary widely. The zinc compound may comprise zinc oxide, zinc ammonium adipate, zinc acetate, zinc ammonium carbonate, zinc stearate, zinc phenyl phosphinic acid, or zinc pyrithione, or combinations thereof. In some embodiments, the zinc compound comprises zinc oxide, zinc ammonium adipate, zinc acetate, or zinc pyrithione, or combinations thereof. In some embodiments, the zinc compound comprises zinc oxide, zinc stearate, or zinc ammonium adipate, or combinations thereof. In some aspects, the zinc is provided in the form of zinc oxide.

In some aspects, the zinc is not provided via zinc phenyl phosphinate and/or zinc phenyl phosphonate.

The inventors have also found that the polymer compositions surprisingly may benefit from the use of specific zinc compounds. In particular, the use of zinc compounds prone to forming ionic zinc (e.g., Zn²⁺) may increase the antiviral properties of the polymer composition. It is theorized that the ionic zinc disrupts the replicative cycle of the virus. For example, the ionic zinc may interfere with, e.g., inhibit viral protease or polymerase activity. Further discussion of the effect of ionic zinc on viral activity is found in Velthuis et al., Zn Inhibits Coronavirus and Arterivirus RNA Polymerase Activity In Vitro and Zinc Ionophores Block the Replication of These Viruses in Cell Culture, PLoS Pathogens (November 2010), which is incorporated herein by reference.

The amount of the zinc compound present in the polymer compositions may be discussed in relation to the ionic zinc content. In one embodiment, the polymer composition comprises ionic zinc, e.g., Zn²+, in an amount ranging from 1 wppm to 30,000 wppm, e.g., from 1 wppm to 25,000 wppm, from 1 wppm to 20,000 wppm, from 1 wppm to 15,000 wppm, from 1 wppm to 10,000 wppm, from 1 wppm to 5,000 wppm, from 1 wppm to 2,500 wppm, from 50 wppm to 30,000 wppm, from 50 wppm to 25,000 wppm, from 50 wppm to 20,000 wppm, from 50 wppm to 15,000 wppm, from 50 wppm to 10,000 wppm, from 50 wppm to 5,000 wppm, from 50 wppm to 2,500 wppm, from 100 wppm to 30,000 wppm, from 100 wppm to 25,000 wppm, from 100 wppm to 20,000 wppm, from 100 wppm to 15,000 wppm, from 100 wppm to 10,000 wppm, from 100 wppm to 5,000 wppm, from 100 wppm to 2,500 wppm, from 150 wppm to 30,000 wppm, from 150 wppm to 25,000 wppm, from 150 wppm to 20,000 wppm, from 150 wppm to 15,000 wppm, from 150 wppm to 10,000 wppm, from 150 wppm to 5,000 wppm, from 150 wppm to 2,500 wppm, from 250 wppm to 30,000 wppm, from 250 wppm to 25,000 wppm, from 250 wppm to 20,000 wppm, from 250 wppm to 15,000 wppm, from 250 wppm to 10,000 wppm, from 250 wppm to 5,000 wppm, or from 250 wppm to 2,500 wppm. In some cases, the ranges and limits mentioned above for zinc may also be applicable to ionic zinc content.

In some cases, the use of zinc provides for processing and or end use benefits. Other antiviral agents, e.g., copper or silver, may be used, but these often include adverse effects (e.g., on the relative viscosity of the polymer composition, toxicity, and health or environmental risk). In some situations, the zinc does not have adverse effects on the relative viscosity of the polymer composition. Also, the zinc, unlike other antiviral agents, e.g., silver, does not present toxicity issues (and in fact may provide health advantages, such as immune system support). In addition, as noted herein, the use of zinc provides for the reduction or elimination of leaching into other media and/or into the environment. This both prevents the risks associated with introducing zinc into the environment and allows the polymer composition to be reused—zinc provides surprising “green” advantages over conventional, e.g., silver-containing, compositions.

As noted above, the polymer composition, in some embodiments, includes copper (provided via a copper compound). As used herein, “copper compound” refers to a compound having at least one copper molecule or ion.

In some cases, the copper compound may improve, e.g., enhance the antiviral properties of the polymer composition. In some cases, the copper compound may affect other characteristics of the polymer composition, e.g., antimicrobial activity or physical characteristics.

The polymer composition may comprise copper (e.g., in a copper compound), e.g., copper or a copper compound, dispersed within the polymer composition. In one embodiment, the polymer composition comprises copper in an amount ranging from 5 wppm to 20,000 wppm, e.g., from 5 wppm to 17,500 wppm, from 5 wppm to 17,000 wppm, from 5 wppm to 16,500 wppm, from 5 wppm to 16,000 wppm, from 5 wppm to 15,500 wppm, from 5 wppm to 15,000 wppm, from 5 wppm to 12,500 wppm, from 5 wppm to 10,000 wppm, from 5 wppm to 5000 wppm, from 5 wppm to 4000 wppm, e.g., from 5 wppm to 3000 wppm, from 5 wppm to 2000 wppm, from 5 wppm to 1000 wppm, from 5 wppm to 500 wppm, from 5 wppm to 100 wppm, from 5 wppm to 50 wppm, from 5 wppm to 35 wppm, from 10 wppm to 20,000 wppm, from 10 wppm to 17,500 wppm, from 10 wppm to 17,000 wppm, from 10 wppm to 16,500 wppm, from 10 wppm to 16,000 wppm, from 10 wppm to 15,500 wppm, from 10 wppm to 15,000 wppm, from 10 wppm to 12,500 wppm, from 10 wppm to 10,000 wppm, from 10 wppm to 5000 wppm, from 10 wppm to 4000 wppm, from 10 wppm to 3000 wppm, from 10 wppm to 2000 wppm, from 10 wppm to 1000 wppm, from 10 wppm to 500 wppm, from 50 wppm to 20,000 wppm, from 50 wppm to 17,500 wppm, from 50 wppm to 17,000 wppm, from 50 wppm to 16,500 wppm, from 50 wppm to 16,000 wppm, from 50 wppm to 15,500 wppm, from 50 wppm to 15,000 wppm, from 50 wppm to 12,500 wppm, from 50 wppm to 10,000 wppm, from 50 wppm to 5000 wppm, from 50 wppm to 4000 wppm, from 50 wppm to 3000 wppm, from 50 wppm to 2000 wppm, from 50 wppm to 1000 wppm, from 50 wppm to 500 wppm, from 100 wppm to 20,000 wppm, from 100 wppm to 17,500 wppm, from 100 wppm to 17,000 wppm, from 100 wppm to 16,500 wppm, from 100 wppm to 16,000 wppm, from 100 wppm to 15,500 wppm, from 100 wppm to 15,000 wppm, from 100 wppm to 12,500 wppm, from 100 wppm to 10,000 wppm, from 100 wppm to 5000 wppm, from 100 wppm to 4000 wppm, from 100 wppm to 3000 wppm, from 100 wppm to 2000 wppm, from 100 wppm to 1000 wppm, from 100 wppm to 500 wppm, from 200 wppm to 20,000 wppm, from 200 wppm to 17,500 wppm, from 200 wppm to 17,000 wppm, from 200 wppm to 16,500 wppm, from 200 wppm to 16,000 wppm, from 200 wppm to 15,500 wppm, from 200 wppm to 15,000 wppm, from 200 wppm to 12,500 wppm, from 200 wppm to 10,000 wppm, from 200 wppm to 5000 wppm, from 200 wppm to 4000 wppm, from 200 wppm to 3000 wppm, from 200 wppm to 2000 wppm, from 200 wppm to 1000 wppm, or from 200 wppm to 500 wppm.

In terms of lower limits, the polymer composition may comprise greater than 5 wppm of copper, e.g., greater than 10 wppm, greater than 50 wppm, greater than 100 wppm, greater than 200 wppm, or greater than 300 wppm. In terms of upper limits, the polymer composition may comprise less than 20,000 wppm of copper, e.g., less than 17,500 wppm, less than 17,000 wppm, less than 16,500 wppm, less than 16,000 wppm, less than 15,500 wppm, less than 15,000 wppm, less than 12,500 wppm, less than 10,000 wppm, less than 5000 wppm, less than less than 4000 wppm, less than 3000 wppm, less than 2000 wppm, less than 1000 wppm, less than 500 wppm less than 100 wppm, less than 50 wppm, less than 35 wppm. In some aspects, the copper compound is embedded in the polymer formed from the polymer composition.

The composition of the copper compound is not particularly limited. Suitable copper compounds include copper iodide, copper bromide, copper chloride, copper fluoride, copper oxide, copper stearate, copper ammonium adipate, copper acetate, or copper pyrithione, or combinations thereof. The copper compound may comprise copper oxide, copper ammonium adipate, copper acetate, copper ammonium carbonate, copper stearate, copper phenyl phosphinic acid, or copper pyrithione, or combinations thereof. In some embodiments, the copper compound comprises copper oxide, copper ammonium adipate, copper acetate, or copper pyrithione, or combinations thereof. In some embodiments, the copper compound comprises copper oxide, copper stearate, or copper ammonium adipate, or combinations thereof. In some aspects, the copper is provided in the form of copper oxide. In some aspects, the copper is not provided via copper phenyl phosphinate and/or copper phenyl phosphonate.

In some cases, the polymer composition includes silver (optionally provided via a silver compound). As used herein, “silver compound” refers to a compound having at least one silver molecule or ion. The silver may be in ionic form. The ranges and limits for silver may be similar to the ranges and limits for copper (discussed above).

In one embodiment, the molar ratio of the copper to the zinc is greater than 0.01:1, e.g., greater than 0.05:1, greater than 0.1:1, greater than 0.15:1, greater than 0.25:1, greater than 0.5:1, or greater than 0.75:1. In terms of ranges, the molar ratio of the copper to the zinc in the polymer composition may range from 0.01:1 to 15:1, e.g., from 0.05:1 to 10:1, from 0.1:1 to 9:1, from 0.15:1 to 8:1, from 0.25:1 to 7:1, from 0.5:1 to 6:1, from 0.75:1 to 5:1 from 0.5:1 to 4:1, or from 0.5:1 to 3:1. In terms of upper limits, the molar ratio of zinc to copper in the polymer composition may be less than 15:1, e.g., less than 10:1, less than 9:1, less than 8:1, less than 7:1, less than 6:1, less than 5:1, less than 4:1, or less than 3:1. In some cases, copper is bound in the polymer matrix along with zinc.

In some embodiments, the use of cuprous ammonium adipate has been found to be particularly effective in activating copper ions into the polymer matrix. Similarly, the use of silver ammonium adipate has been found to be particularly effective in activating silver ions into the polymer matrix. It is found that dissolving copper (I) or copper (II) compounds in ammonium adipate is particularly efficient at generating copper (I) or copper (II) ions. The same is true for dissolving Ag (I) or Ag (III) compounds in ammonium adipate to generate Ag1+ or Ag3+ ions.

The polymer composition may comprise silver (e.g., in a silver compound), e.g., silver or a silver compound, dispersed within the polymer composition. In one embodiment, the polymer composition comprises silver in an amount ranging from 5 wppm to 20,000 wppm, e.g., from 5 wppm to 17,500 wppm, from 5 wppm to 17,000 wppm, from 5 wppm to 16,500 wppm, from 5 wppm to 16,000 wppm, from 5 wppm to 15,500 wppm, from 5 wppm to 15,000 wppm, from 5 wppm to 12,500 wppm, from 5 wppm to 10,000 wppm, from 5 wppm to 5000 wppm, from 5 wppm to 4000 wppm, e.g., from 5 wppm to 3000 wppm, from 5 wppm to 2000 wppm, from 5 wppm to 1000 wppm, from 5 wppm to 500 wppm, from 10 wppm to 20,000 wppm, from 10 wppm to 17,500 wppm, from 10 wppm to 17,000 wppm, from 10 wppm to 16,500 wppm, from 10 wppm to 16,000 wppm, from 10 wppm to 15,500 wppm, from 10 wppm to 15,000 wppm, from 10 wppm to 12,500 wppm, from 10 wppm to 10,000 wppm, from 10 wppm to 5000 wppm, from 10 wppm to 4000 wppm, from 10 wppm to 3000 wppm, from 10 wppm to 2000 wppm, from 10 wppm to 1000 wppm, from 10 wppm to 500 wppm, from 50 wppm to 20,000 wppm, from 50 wppm to 17,500 wppm, from 50 wppm to 17,000 wppm, from 50 wppm to 16,500 wppm, from 50 wppm to 16,000 wppm, from 50 wppm to 15,500 wppm, from 50 wppm to 15,000 wppm, from 50 wppm to 12,500 wppm, from 50 wppm to 10,000 wppm, from 50 wppm to 5000 wppm, from 50 wppm to 4000 wppm, from 50 wppm to 3000 wppm, from 50 wppm to 2000 wppm, from 50 wppm to 1000 wppm, from 50 wppm to 500 wppm, from 100 wppm to 20,000 wppm, from 100 wppm to 17,500 wppm, from 100 wppm to 17,000 wppm, from 100 wppm to 16,500 wppm, from 100 wppm to 16,000 wppm, from 100 wppm to 15,500 wppm, from 100 wppm to 15,000 wppm, from 100 wppm to 12,500 wppm, from 100 wppm to 10,000 wppm, from 100 wppm to 5000 wppm, from 100 wppm to 4000 wppm, from 100 wppm to 3000 wppm, from 100 wppm to 2000 wppm, from 100 wppm to 1000 wppm, from 100 wppm to 500 wppm, from 200 wppm to 20,000 wppm, from 200 wppm to 17,500 wppm, from 200 wppm to 17,000 wppm, from 200 wppm to 16,500 wppm, from 200 wppm to 16,000 wppm, from 200 wppm to 15,500 wppm, from 200 wppm to 15,000 wppm, from 200 wppm to 12,500 wppm, from 200 wppm to 10,000 wppm, from 200 wppm to 5000 wppm, from 200 wppm to 4000 wppm, from 200 wppm to 3000 wppm, from 200 wppm to 2000 wppm, from 200 wppm to 1000 wppm, or from 200 wppm to 500 wppm.

In terms of lower limits, the polymer composition may comprise greater than 5 wppm of silver, e.g., greater than 10 wppm, greater than 50 wppm, greater than 100 wppm, greater than 200 wppm, or greater than 300 wppm. In terms of upper limits, the polymer composition may comprise less than 20,000 wppm of silver, e.g., less than 17,500 wppm, less than 17,000 wppm, less than 16,500 wppm, less than 16,000 wppm, less than 15,500 wppm, less than 15,000 wppm, less than 12,500 wppm, less than 10,000 wppm, less than 5000 wppm, less than less than 4000 wppm, less than 3000 wppm, less than 2000 wppm, less than 1000 wppm, or less than 500 wppm. In some aspects, the silver compound is embedded in the polymer formed from the polymer composition.

The composition of the silver compound is not particularly limited. Suitable silver compounds include silver iodide, silver bromide, silver chloride, silver fluoride, silver oxide, silver stearate, silver ammonium adipate, silver acetate, or silver pyrithione, or combinations thereof. The silver compound may comprise silver oxide, silver ammonium adipate, silver acetate, silver ammonium carbonate, silver stearate, silver phenyl phosphinic acid, or silver pyrithione, or combinations thereof. In some embodiments, the silver compound comprises silver oxide, silver ammonium adipate, silver acetate, or silver pyrithione, or combinations thereof. In some embodiments, the silver compound comprises silver oxide, silver stearate, or silver ammonium adipate, or combinations thereof. In some aspects, the silver is provided in the form of silver oxide. In some aspects, the silver is not provided via silver phenyl phosphinate and/or silver phenyl phosphonate. In some aspects, the silver is provided by dissolving one or more silver compounds in ammonium adipate.

The polymer composition may comprise phosphorus (in a phosphorus compound), e.g., phosphorus or a phosphorus compound is dispersed within the polymer composition. In one embodiment, the polymer composition comprises phosphorus in an amount ranging from 50 wppm to 10000 wppm, e.g., from 50 wppm to 5000 wppm, from 50 wppm to 2500 wppm, from 50 wppm to 2000 wppm, from 50 wppm to 800 wppm, 100 wppm to 750 wppm, 100 wppm to 1800 wppm, from 100 wppm to 10000 wppm, from 100 wppm to 5000 wppm, from 100 wppm to 2500 wppm, from 100 wppm to 1000 wppm, from 100 wppm to 800 wppm, from 200 wppm to 10000 wppm, 200 wppm to 5000 wppm, from 200 wppm to 2500 wppm, from 200 wppm to 800 wppm, from 300 wppm to 10000 wppm, from 300 wppm to 5000 wppm, from 300 wppm to 2500 wppm, from 300 wppm to 500 wppm, from 500 wppm to 10000 wppm, from 500 wppm to 5000 wppm, or from 500 wppm to 2500 wppm. In terms of lower limits, the polymer composition may comprise greater than 50 wppm of phosphorus, e.g., greater than 75 wppm, greater than 100 wppm, greater than 150 wppm, greater than 200 wppm greater than 300 wppm or greater than 500 wppm. In terms of upper limits, the polymer composition may comprise less than 10000 wppm (or 1 wt. %), e.g., less than 5000 wppm, less than 2500 wppm, less than 2000 wppm, less than 1800 wppm, less than 1500 wppm, less than 1000 wppm, less than 800 wppm, less than 750 wppm, less than 500 wppm, less than 475 wppm, less than 450 wppm, less than 400 wppm, less than 350 wppm, less than 300 wppm, less than 250 wppm, less than 200 wppm, less than 150 wppm, less than 100 wppm, less than 50 wppm, less than 25 wppm, or less than 10 wppm.

In some aspects, the phosphorus or the phosphorus compound is embedded in the polymer formed from the polymer composition. As noted above, because of the overall make-up of the disclosed composition low amounts, if any, phosphorus may be employed, which in some cases may provide for advantageous performance results (see above).

The phosphorus of the polymer composition is present in or provided via a phosphorus compound, which may vary widely. The phosphorus compound may comprise benzene phosphinic acid, diphenylphosphinic acid, sodium phenylphosphinate, phosphorous acid, benzene phosphonic acid, calcium phenylphosphinate, potassium B-pentylphosphinate, methylphosphinic acid, manganese hypophosphite, sodium hypophosphite, monosodium phosphate, hypophosphorous acid, dimethylphosphinic acid, ethylphosphinic acid, diethylphosphinic acid, magnesium ethylphosphinate, triphenyl phosphite, diphenylmethyl phosphite, dimethylphenyl phosphite, ethyldiphenyl phosphite, phenylphosphonic acid, methylphosphonic acid, ethylphosphonic acid, potassium phenylphosphonate, sodium methylphosphonate, calcium ethylphosphonate, and combinations thereof. In some embodiments, the phosphorus compound comprises phosphoric acid, benzene phosphinic acid, or benzene phosphonic acid, or combinations thereof. In some embodiments, the phosphorus compound comprises benzene phosphinic acid, phosphorous acid, or manganese hypophosphite, or combinations thereof. In some aspects, the phosphorus compound may comprise benzene phosphinic acid.

In one embodiment, the molar ratio of the phosphorus to the zinc is greater than 0.01:1, e.g., greater than 0.05:1, greater than 0.1:1, greater than 0.15:1, greater than 0.25:1, greater than 0.5:1, or greater than 0.75:1. In terms of ranges, the molar ratio of the phosphorus to the zinc in the polymer composition may range from 0.01:1 to 15:1, e.g., from 0.05:1 to 10:1, from 0.1:1 to 9:1, from 0.15:1 to 8:1, from 0.25:1 to 7:1, from 0.5:1 to 6:1, from 0.75:1 to 5:1 from 0.5:1 to 4:1, or from 0.5:1 to 3:1. In terms of upper limits, the molar ratio of zinc to phosphorus in the polymer composition may be less than 15:1, e.g., less than 10:1, less than 9:1, less than 8:1, less than 7:1, less than 6:1, less than 5:1, less than 4:1, or less than 3:1. In some cases, phosphorus is bound in the polymer matrix along with zinc.

In one embodiment, the weight ratio of zinc to phosphorus in the polyamide composition may be greater than 1.3:1, e.g., greater than 1.4:1, greater than 1.5:1, greater than 1.6:1, greater than 1.7:1, greater than 1.8:1, or greater than 2:1. In terms of ranges, the weight ratio of zinc to phosphorus in the polyamide composition may range from 1.3:1 to 30:1, e.g., from 1.4:1 to 25:1, from 1.5:1 to 20:1, from 1.6:1 to 15:1, from 1.8:1 to 10:1, from 2:1 to 8:1, from 3:1 to 7:1, or from 4:1 to 6:1. In terms of upper limits, the weight ratio of zinc to phosphorus in the polyamide composition may be less than 30:1, e.g., less than 28:1, less than 26:1, less than 24:1, less than 22:1, less than 20:1, or less than 15:1. In some aspects, there is no phosphorus in the polyamide composition. In other aspects, a very low amount of phosphorus is present. In some cases, phosphorus is held in the polymer matrix along with zinc.

In one embodiment, the weight ratio of zinc to phosphorus in the polyamide composition may be less than 0.64:1, e.g., less than 0.62:1, less than 0.6:1, e.g., less than 0.5:1, less than 0.45:1, less than 0.4:1, less than 0.3:1, or less than 0.25:1. In terms of ranges, the weight ratio of zinc to phosphorus in the polyamide composition may range from 0.001:1 to 0.64:1, e.g., from 0.01:1 to 0.6:1, from 0.05:1 to 0.5:1, from 0.1:1 to 0.45:1, from 0.2:1 to 0.4:1, from 0.25:1 to 0.35:1, or from 0.2:1 to 0.3:1. In terms of lower limits, the weight ratio of zinc to phosphorus in the polyamide composition may be greater than 0.001:1, e.g., greater than 0.005:1, greater than 0.01:1, greater than 0.05:1, greater than 0.1:1, greater than 0.15:1, or greater than 0.2:1.

Advantageously, it has been discovered that adding the above identified zinc compounds and phosphorus compounds may result in a beneficial relative viscosity (RV) of the polymer composition. In some embodiments, the RV of the polymer composition ranges from 5 to 80, e.g., from 5 to 70, from 10 to 70, from 15 to 65, from 20 to 60, from 30 to 50, from 10 to 35, from 10 to 20, from 60 to 70, from 50 to 80, from 40 to 50, from 30 to 60, from 5 to 30, or from 15 to 32. In terms of lower limits, the RV of the polymer composition may be greater than 5, e.g., greater than 10, greater than 15, greater than 20, greater than 25, greater than 27.5, or greater than 30. In terms of upper limits, the RV of the polymer composition may be less than 70, e.g., less than 65, less than 60, less than 50, less than 40, or less than 35.

To calculate RV, a polymer is dissolved in a solvent (usually formic or sulfuric acid), the viscosity is measured, then the viscosity is compared to the viscosity of the pure solvent. This give a unitless measurement. Solid materials, as well as liquids, may have a specific RV. The fibers/fabrics produced from the polymer compositions may have the aforementioned relative viscosities, as well.

It has been determined that a specific amount of the zinc compound and the phosphorus compound can be mixed in a polymer composition, e.g., polyamide composition, in finely divided form, such as in the form of granules, flakes and the like, to provide a polymer composition that can be subsequently formed, e.g., extruded, molded or otherwise drawn, into various products (e.g., high-contact products, surtopsheet layers of high-contact products) by conventional methods to produce products having substantially improved antimicrobial activity. The zinc and phosphorus are employed in the polymer composition in the aforementioned amounts to provide a fiber with improved antimicrobial activity retention (near-permanent).

### Additional Components

In some embodiments, the polymer composition may comprise additional additives. The additives include pigments, hydrophilic or hydrophobic additives, anti-odor additives, additional antiviral agents, and antimicrobial/anti-fungal inorganic compounds, such as copper, zinc, tin, and silver.

In some embodiments, the polymer composition can be combined with color pigments for coloration for the use in fabrics or other components formed from the polymer composition. In some aspects, the polymer composition can be combined with UV additives to withstand fading and degradation in fabrics exposed to significant UV light. In some aspects, the polymer composition can be combined with additives to make the surface of the fiber hydrophilic or hydrophobic. In some aspects, the polymer composition can be combined with a hygroscopic material, e.g., to make the fiber, fabric, or other products formed therefrom more hygroscopic. In some aspects, the polymer composition can be combined with additives to make the fabric flame retardant or flame resistant. In some aspects, the polymer composition can be combined with additives to make the fabric stain resistant. In some aspects, the polymer composition can be combined with pigments with the antimicrobial compounds so that the need for conventional dyeing and disposal of dye materials is avoided.

In some embodiments, the polymer composition may further comprise additional additives. For example, the polymer composition may comprise a delusterant. A delusterant additive may improve the appearance and/or texture of the synthetic fibers and fabric produced from the polymer composition. In some embodiments, inorganic pigment-like materials can be utilized as delusterants. The delusterants may comprise one or more of titanium dioxide, barium sulfate, barium titanate, zinc titanate, magnesium titanate, calcium titanate, zinc oxide, zinc sulfide, lithopone, zirconium dioxide, calcium sulfate, barium sulfate, aluminum oxide, thorium oxide, magnesium oxide, silicon dioxide, talc, mica, and the like. In preferred embodiments, the delusterant comprises titanium dioxide. It has been found that the polymer compositions that include delusterants comprising titanium dioxide produce synthetic fibers and fabrics that greatly resemble natural fibers and fabrics, e.g., synthetic fibers and fabrics with improved appearance and/or texture. It is believed that titanium dioxide improves appearance and/or texture by interacting with the zinc compound, the phosphorus compound, and/or functional groups within the polymer.

In one embodiment, the polymer composition comprises the delusterant in an amount ranging from 0.0001 wt. % to 3 wt. %, e.g., 0.0001 wt. % to 2 wt. %, from 0.0001 to 1.75 wt. %, from 0.001 wt. % to 3 wt. %, from 0.001 wt. % to 2 wt. %, from 0.001 wt. % to 1.75 wt. %, from 0.002 wt. % to 3 wt. %, from 0.002 wt. % to 2 wt. %, from 0.002 wt. % to 1.75 wt. %, from 0.005 wt. % to 3 wt. %, from 0.005 wt. % to 2 wt. %, from 0.005 wt. % to 1.75 wt. %. In terms of upper limits, the polymer composition may comprise less than 3 wt. % delusterant, e.g., less than 2.5 wt. %, less than 2 wt. % or less than 1.75 wt. %. In terms of lower limits, the polymer composition may comprise greater than 0.0001 wt. % delusterant, e.g., greater than 0.001 wt. %, greater than 0.002 wt. %, or greater than 0.005 wt. %.

In some embodiments, the polymer composition may further comprise colored materials, such as carbon black, copper phthalocyanine pigment, lead chromate, iron oxide, chromium oxide, and ultramarine blue.

In some embodiments, the polymer composition may include additional antiviral agents other than zinc. The additional antimicrobial agents may be any suitable antiviral. Conventional antiviral agents are known in the art and may be incorporated in the polymer composition as the additional antiviral agent or agents. For example, the additional antiviral agent may be an entry inhibitor, a reverse transcriptase inhibitor, a DNA polymerase inhibitor, an m-RNA synthesis inhibitor, a protease inhibitor, an integrase inhibitor, or an immunomodulator, or combinations thereof. In some aspects, the additional antimicrobial agent or agents are added to the polymer composition.

In some embodiments, the polymer composition may include additional antimicrobial agents other than zinc. The additional antimicrobial agents may be any suitable antimicrobial, such as silver, copper, and/or gold in metallic forms (e.g., particulates, alloys and oxides), salts (e.g., sulfates, nitrates, acetates, citrates, and chlorides) and/or in ionic forms. In some aspects, further additives, e.g., additional antimicrobial agents, are added to the polymer composition.

In some embodiments, the polymer composition (and the fibers or fabric formed therefrom) may further comprise an antimicrobial or antiviral coating. For example, a fiber or fabric formed from the polymer composition may include a coating of zinc nanoparticles (e.g., nanoparticles of zinc oxide, zinc ammonium adipate, zinc acetate, zinc ammonium carbonate, zinc stearate, zinc phenyl phosphinic acid, or zinc pyrithione, or combinations thereof). To produce such a coating, the surface of polymer composition (e.g., the surface of the fiber and/or fabric formed therefrom) may be cationized and coated layer-by layer by stepwise dipping the polymer composition into an anionic polyelectrolyte solution (e.g., comprising poly 4-styrenesulfonic acid) and a solution comprising the zinc nanoparticles. Optionally, the coated polymer composition may be hydrothermally treated in a solution of NH₄OH at 9° C. for 24 h to immobilize the zinc nanoparticles.

In some cases, the AM/AV materials described herein do not require the use or inclusion of acids, e.g., citric acid, and/or acid treatment to be effective. Such treatments are known to create static charge/static decay issues. Advantageously, the elimination of the need for acid treatment, thus eliminates the static charge/static decay issues associated with conventional configurations.

In some embodiments, any or some of the components disclosed herein may be considered optional. In some cases, the disclosed compositions may expressly exclude any or some of the aforementioned additives in this description, e.g., via claim language. For example claim language may be modified to recite that the disclosed compositions, materials processes, etc., do not utilize or comprise one or more of the aforementioned additives, e.g., the disclosed materials do not comprise a flame retardant or a delusterant. As another example, the claim language may be modified to recite that the disclosed materials do not comprise long chain polyamide component, e.g., PA-12.

### Metal Retention Rate

As noted, the AM/AV materials described herein have permanent, e.g., near-permanent, antimicrobial and/or antiviral properties. The permanence of these properties allows the AM/AV materials to be reused, e.g., after washing, further extending the usefulness of the article.

One metric for assessing the permanence, e.g., near-permanence, of the antimicrobial and/or antiviral properties of the AM/AV material is metal retention. As discussed above, the AM/AV materials may be prepared from the disclosed polymer compositions, which may include various metallic compounds, e.g., zinc compound, phosphorus, copper compound, and/or silver compound. The metallic compounds of the polymer compositions may provide antimicrobial and/or antiviral properties to the AM/AV material. Thus, retention of the metallic compounds, e.g., after one or more cycles of washing, may provide permanent, e.g., near-permanent, antimicrobial and/or antiviral properties.

Beneficially, AM/AV materials formed from the disclosed polymer compositions demonstrate relatively high metal retention rate. The metal retention rate may relate to the retention rate of a specific metal in the polymer composition, e.g., zinc retention, copper retention, or to the retention rate of all metals in the polymer composition, e.g., total metal retention.

In some embodiments, the AM/AV materials formed from the disclosed polymer compositions have a metal retention greater than 65% as measured by a dye bath test, e.g., greater than 75%, greater than 80%, greater than 90%, greater than 95%, greater than 97%, greater than 98%, greater than 99%, greater than 99.9%, greater than 99.99%, greater than 99.999%, greater than 99.9999%, greater than 99.99999% or greater than 99.999999%. In terms of upper limits, the AM/AV materials may have a metal retention of less than 100%, e.g., less than 99.9%, less than 98%, or less than 95%. In terms of ranges, the AM/AV materials may have a metal retention may be from 60% to 100%, e.g., from 60% to 99.999999%, from 60% to 99.99999%, from 60% to 99.9999%, from 60% to 99.999% from 60% to 99.999%, from 60% to 99.99%, from 60% to 99.9%, from 60% to 99%, from 60% to 98%, from 60% to 95%, from 65% to 99.999999%, from 65% to 99.99999%, from 65% to 99.9999%, from 65% to 99.999% from 65% to 99.999%, from 65% to 100%, from 65% to 99.99%, from 65% to 99.9%, from 65% to 99%, from 65% to 98%, from 65% to 95%, from 70% to 100%, from 70% to 99.999999%, from 70% to 99.99999%, from 70% to 99.9999%, from 70% to 99.999% from 70% to 99.999%, from 70% to 99.99%, from 70% to 99.9%, from 70% to 99%, from 70% to 98%, from 70% to 95%, from 75% to 100%, from 75% to 99.99%, from 75% to 99.9%, from 75% to 99.999999%, from 75% to 99.99999%, from 75% to 99.9999%, from 75% to 99.999% from 75% to 99.999%, from 75% to 99%, from 75% to 98%, from 75% to 95%, %, from 80% to 99.999999%, from 80% to 99.99999%, from 80% to 99.9999%, from 80% to 99.999% from 80% to 99.999%, from 80% to 100%, from 80% to 99.99%, from 80% to 99.9%, from 80% to 99%, from 80% to 98%, or from 80% to 95%. In some cases, the ranges and limits relate to dye recipes having lower pH values, e.g., less than (and/or including) 5.0, less than 4.7, less than 4.6, or less than 4.5. In some cases, the ranges and limits relate to dye recipes having higher pH values, e.g., greater than (and/or including) 4.0, greater than 4.2, greater than 4.5, greater than 4.7, greater than 5.0, or greater than 5.2.

In some embodiments, the AM/AV materials formed from the disclosed polymer compositions have a metal retention greater than 40% after a dye bath, e.g., greater than 44%, greater than 45%, greater than 50%, greater than 55%, greater than 60%, greater than 65%, greater than 70%, greater than 75%, greater than 80%, greater than 90%, greater than 95%, or greater than 99%. In terms of upper limits, the AM/AV materials may have a metal retention of less than 100%, e.g., less than 99.9%, less than 98%, less than 95% or less than 90%. In terms of ranges, the AM/AV materials may have a metal retention in a range from 40% to 100%, e.g., from 45% to 99.9%, from 50% to 99.9%, from 75% to 99.9%, from 80% to 99%, or from 90% to 98%. In some cases, the ranges and limits relate to dye recipes having higher pH values, e.g., greater than (and/or including) 4.0, greater than 4.2, greater than 4.5, greater than 4.7, greater than 5.0, or greater than 5.2.

In some embodiments, the AM/AV materials formed from the polymer compositions have a metal retention greater than 20%, e.g., greater than 24%, greater than 25%, greater than 30%, greater than 35%, greater than 40%, greater than 45%, greater than 50%, greater than 55%, or greater than 60%. In terms of upper limits, the AM/AV materials may have a metal retention of less than 80%, e.g., less than 77%, less than 75%, less than 70%, less than 68%, or less than 65%. In terms of ranges, the AM/AV materials may have a metal retention ranging from 20% to 80%, e.g., from 25% to 77%, from 30% to 75%, or from 35% to 70%. In some cases, the ranges and limits relate to dye recipes having lower pH values, e.g., less than (and/or including) 5.0, less than 4.7, less than 4.6, or less than 4.5.

Stated another way, in some embodiments, the AM/AV materials formed from the polymer composition demonstrate an extraction rate of the metal compound less than 35% as measured by the dye bath test, e.g., less than 25%, less than 20%, less than 10%, or less than 5%. In terms of upper limits, the AM/AV materials may demonstrate an extraction rate of the metal compound greater than 0%, e.g., greater than 0.1%, greater than 2% or greater than 5%. In terms of ranges, the AM/AV materials may demonstrate an extraction rate of the metal compound from 0% to 35%, e.g., from 0% to 25%, from 0% to 20%, from 0% to 10%, from 0% to 5%, from 0.1% to 35%, from 0.1% to 25%, from 0.1% to 20%, from 0.2% to 10%, from 0.1% to 5%, from 2% to 35%, from 2% to 25%, from 2% to 20%, from 2% to 10%, from 2% to 5%, from 5% to 35%, from 5% to 25%, from 5% to 20%, or from 5% to 10%.

The metal retention of a AM/AV material may be measured by a dye bath test according to the following standard procedure. A sample is cleaned (all oils are removed) by a scour process. The scour process may employ a heated bath, e.g., conducted at 71° C. for 15 minutes. A scouring solution comprising 0.25% on weight of fiber (“owf”) of Sterox (723 Soap) nonionic surfactant and 0.25% owf of TSP (trisodium phosphate) may be used. The samples are then rinsed with cold water.

The cleaned samples may be tested according a chemical dye level procedure. This procedure may employ placing them in a dye bath comprising 1.0% owf of C.I. Acid Blue 45, 4.0% owf of MSP (monosodium phosphate), and a sufficient % owf of disodium phosphate or TSP to achieve a pH of 6.0, with a 28:1 liquor to sample ratio. For example, if a pH of less than 6 is desired, a 10% solution of the desired acid may be added using an eye dropper until the desired pH was achieved. The dye bath may be preset to bring the bath to a boil at 100° C. The samples are placed in the bath for 1.5 hours. As one example, it may take approximately 30 minutes to reach boil and hold one hour after boil at this temperature. Then the samples are removed from the bath and rinsed. The samples are then transferred to a centrifuge for water extraction. After water extraction, the samples were laid out to air dry. The component amounts are then recorded.

In some embodiments, the metal retention of a fiber formed from the polymer composition may be calculated by measuring metal content before and after a dye bath operation. The amount of metal retained after the dye bath may be measured by known methods. For the dye bath, an Ahiba dyer (from Datacolor) may be employed. In a particular instance, twenty grams of un-dyed fabric and 200 ml of dye liquor may be placed in a stainless steel can, the pH may be adjusted to the desired level, the stainless steel can may be loaded into the dyer; the sample may be heated to 40° C. then heated to 100° C. (optionally at 1.5° C./minute). In some cases a temperature profile may be employed, for example, 1.5° C./minute to 60° C., 1° C./minute to 80° C., and 1.5° C./minute to 100° C. The sample may be held at 100° C. for 45 minutes, followed by cooling to 40° C. at 2° C./minute, then rinsed and dried to yield the dyed product.

In some embodiments, the AM/AV material, e.g., one or more layers of the AM/AV material, retains AM/AV properties after one or more washing cycles. In some cases, this washfastness may be due to the use of the aforementioned AM/AV formulations employed to make the fibers/fabrics, e.g., the AM/AV compound may be embedded in the polymer structure. In one embodiment, the AM/AV material retains AM/AV properties after more than 1 washing cycle, e.g., more than 2 washing cycles, more than 5 washing cycles, more than 10 washing cycles, or more than 20 washing cycle. The durability of the disclosed materials and/or layers is also demonstrated via retention after dyeing operations.

The washfastness may also be described by the metal retention (e.g., zinc retention) after a number of wash cycles. In some embodiments, for example, the AM/AV material retains greater than 95% of a metallic compound (e.g., a zinc compound) after 5 wash cycles, e.g., greater than 96%, greater than 97%, or greater than 98%. In some embodiments, the AM/AV material retains greater than 85% of a metallic compound (e.g., a zinc compound) after 10 wash cycles, e.g., greater than 86%, greater than 87%, greater than 88%, greater than 89%, or greater than 90%.

In some cases, the AM/AV materials may be used in wound care, for example, the AM/AV materials may be employed as wraps, (breathable) gauzes, bandages, and/or other dressings. The AM/AV properties of the AM/AV materials make them particularly beneficial in these applications. In some cases, the AM/AV materials serve as a moisture barrier and/or to facilitate an oxygen transmission balance.

### Method of Forming Fibers and Nonwoven Fabrics

As described herein, the fibers or fabrics of the AM/AV material are made by forming the AM/AV polymer composition into the fibers, which are arranged to form the fabric or structure.

In some aspects, fibers, e.g., polyamide fibers, are made by spinning a polyamide composition formed in a melt polymerization process. During the melt polymerization process of the polyamide composition, an aqueous monomer solution, e.g., salt solution, is heated under controlled conditions of temperature, time and pressure to evaporate water and effect polymerization of the monomers, resulting in a polymer melt. During the melt polymerization process, sufficient amounts of zinc and, optionally, phosphorus, are employed in the aqueous monomer solution to form the polyamide mixture before polymerization. The monomers are selected based on the desired polyamide composition. After zinc and phosphorus are present in the aqueous monomer solution, the polyamide composition may be polymerized. The polymerized polyamide can subsequently be spun into fibers, e.g., by melt, solution, centrifugal, or electro-spinning.

In some embodiments, the process for preparing fibers having permanent AM/AV properties from the polyamide composition includes preparing an aqueous monomer solution, adding less than 20,000 wppm of one or more metallic compounds dispersed within the aqueous monomer solution, e.g., less than 17,500 wppm, less than 17,000 wppm, less than 16,500 wppm, less than 16,000 wppm, less than 15,500 wppm, less than 15,000 wppm, less than 12,500 wppm, less than 10,000 wppm, less than 5000 wppm, less than less than 4000 wppm, less than 3000 wppm, less than 2000 wppm, less than 1000 wppm, or less than 500 wppm, polymerizing the aqueous monomer solution to form a polymer melt, and spinning the polymer melt to form an AM/AV fiber. In this embodiment, the polyamide composition comprises the resultant aqueous monomer solution after the metallic compound(s) are added.

In some embodiments, the process includes preparing an aqueous monomer solution. The aqueous monomer solution may comprise amide monomers. In some embodiments, the concentration of monomers in the aqueous monomer solution is less than 60 wt %, e.g., less than 58 wt %, less than 56.5 wt %, less than 55 wt %, less than 50 wt %, less than 45 wt %, less than 40 wt %, less than 35 wt %, or less than 30 wt %. In some embodiments, the concentration of monomers in the aqueous monomer solution is greater than 20 wt %, e.g., greater than 25 wt %, greater than 30 wt %, greater than 35 wt %, greater than 40 wt %, greater than 45 wt %, greater than 50 wt %, greater than 55 wt %, or greater than 58 wt %. In some embodiments, the concentration of monomers in the aqueous monomer solution is in a range from 20 wt % to 60 wt %, e.g., from 25 wt % to 58 wt %, from 30 wt % to 56.5 wt %, from 35 wt % to 55 wt %, from 40 wt % to 50 wt %, or from 45 wt % to 55 wt %. The balance of the aqueous monomer solution may comprise water and/or additional additives. In some embodiments, the monomers comprise amide monomers including a diacid and a diamine, i.e., nylon salt.

In some embodiments, the aqueous monomer solution is a nylon salt solution. The nylon salt solution may be formed by mixing a diamine and a diacid with water. For example, water, diamine, and dicarboxylic acid monomer are mixed to form a salt solution, e.g., mixing adipic acid and hexamethylene diamine with water. In some embodiments, the diacid may be a dicarboxylic acid and may be selected from the group consisting of oxalic acid, malonic acid, succinic acid, glutaric acid, pimelic acid, adipic acid, suberic acid, azelaic acid, sebacic acid, undecanedioic acid, dodecandioic acid, maleic acid, glutaconic acid, traumatic acid, and muconic acid, 1,2- or 1,3-cyclohexane dicarboxylic acids, 1,2- or 1,3 -phenyl enediacetic acids, 1,2- or 1,3-cyclohexane diacetic acids, isophthalic acid, terephthalic acid, 4,4′-oxybisbenzoic acid, 4,4-benzophenone dicarboxylic acid, 2,6-napthalene dicarboxylic acid, p-t-butyl isophthalic acid and 2,5-furandicarboxylic acid, and mixtures thereof. In some embodiments, the diamine may be selected from the group consisting of ethanol diamine, trimethylene diamine, putrescine, cadaverine, hexamethyelene diamine, 2-methyl pentamethylene diamine, heptamethylene diamine, 2-methyl hexamethylene diamine, 3 -methyl hexamethylene diamine, 2,2-dimethyl pentamethylene diamine, octamethylene diamine, 2,5-dimethyl hexamethylene diamine, nonamethylene diamine, 2,2,4- and 2,4,4-trimethyl hexamethylene diamines, decamethylene diamine, 5-methylnonane diamine, isophorone diamine, undecamethylene diamine, dodecamethylene diamine, 2,2,7,7-tetramethyl octamethylene diamine, bis(p-aminocyclohexyl)methane, bis(aminomethyl)norbornane, C2-C16 aliphatic diamine optionally substituted with one or more C1 to C4 alkyl groups, aliphatic polyether diamines and furanic diamines, such as 2,5-bis(aminomethyl)furan, and mixtures thereof. In preferred embodiments, the diacid is adipic acid and the diamine is hexamethylene diamine which are polymerized to form PA6,6.

It should be understood that the concept of producing a polyamide from diamines and diacids also encompasses the concept of other suitable monomers, such as, aminoacids or lactams. Without limiting the scope, examples of aminoacids can include 6-aminohaxanoic acid, 7-aminoheptanoic acid, 11-aminoundecanoic acid, 12-aminododecanoic acid, or combinations thereof. Without limiting the scope of the disclosure, examples of lactams can include caprolactam, enantholactam, lauryllactam, or combinations thereof. Suitable feeds for the disclosed process can include mixtures of diamines, diacids, aminoacids and lactams.

After the aqueous monomer solution is prepared, a metallic compound (e.g., a zinc compound, a copper compound, and/or a silver compound) is added to the aqueous monomer solution to form the polyamide composition. In some embodiments, less than 20,000 wppm of the metallic compound is dispersed within the aqueous monomer solution. In some aspects, further additives, e.g., additional AM/AV agents, are added to the aqueous monomer solution. Optionally, phosphorus (e.g., a phosphorus compound) is added to the aqueous monomer solution.

In some cases, the polyamide composition is polymerized using a conventional melt polymerization process. In one aspect, the aqueous monomer solution is heated under controlled conditions of time, temperature, and pressure to evaporate water, effect polymerization of the monomers and provide a polymer melt. In some aspects, the particular weight ratio of zinc to phosphorus may advantageously promote binding of zinc within the polymer, reduce thermal degradation of the polymer, and enhance its dyeability.

In one embodiment, a nylon is prepared by a conventional melt polymerization of a nylon salt. Typically, the nylon salt solution is heated under pressure, e.g. 250 psig/1825×10³ n/m²,to a temperature of, for example, about 245° C. Then the water vapor is exhausted off by reducing the pressure to atmospheric pressure while increasing the temperature to, for example, about 270° C. Before polymerization, zinc and, optionally, phosphorus be added to the nylon salt solution. The resulting molten nylon is held at this temperature for a period of time to bring it to equilibrium prior to being extruded into a fiber. In some aspects, the process may be carried out in a batch or continuous process.

In some embodiments, during melt polymerization, zinc, e.g., zinc oxide is added to the aqueous monomer solution. The AM/AV fiber may comprise a polyamide that is made in a melt polymerization process and not in a master batch process. In some aspects, the resulting fiber has permanent AM/AV properties. The resulting fiber can be used in the topsheet layer and/or the pad layer of the AM/AV material.

The AM/AV agent may be added to the polyamide during melt polymerization, for example as a master batch or as a powder added to the polyamide pellets, and thereafter, the fiber may be formed from spinning. The fibers are then formed into a nonwoven structure.

In some aspects, the AM/AV nonwoven structure is melt blown. Melt blowing is advantageously less expensive than electrospinning. Melt blowing is a process type developed for the formation of microfibers and nonwoven webs. Until recently, microfibers have been produced by melt blowing. Now, nanofibers may also be formed by melt blowing. The nanofibers are formed by extruding a molten thermoplastic polymeric material, or polyamide, through a plurality of small holes. The resulting molten threads or filaments pass into converging high velocity gas streams which attenuate or draw the filaments of molten polyamide to reduce their diameters. Thereafter, the melt blown nanofibers are carried by the high velocity gas stream and deposited on a collecting surface, or forming wire, to form a nonwoven web of randomly disbursed melt blown nanofibers. The formation of nanofibers and nonwoven webs by melt blowing is well known in the art. See, e.g., U.S. Pat. Nos. 3,704,198; 3,755,527; 3,849,241; 3,978,185; 4,100,324; and 4,663,220.

One option, “Island-in-the-sea,” refers to fibers forming by extruding at least two polymer components from one spinning die, also referred to as conjugate spinning.

As is well known, electrospinning has many fabrication parameters that may limit spinning certain materials. These parameters include: electrical charge of the spinning material and the spinning material solution; solution delivery (often a stream of material ejected from a syringe); charge at the jet; electrical discharge of the fibrous membrane at the collector; external forces from the electrical field on the spinning jet; density of expelled jet; and (high) voltage of the electrodes and geometry of the collector. In contrast, the aforementioned nanofibers and products are advantageously formed without the use of an applied electrical field as the primary expulsion force, as is required in an electrospinning process. Thus, the polyamide is not electrically charged, nor are any components of the spinning process. Importantly, the dangerous high voltage necessary in electrospinning processes, is not required with the presently disclosed processes/products. In some embodiments, the process is a non-electrospin process and resultant product is a non-electrospun product that is produced via a non-electrospin process.

Another embodiment of making the nanofiber nonwovens is by way of 2-phase spinning or melt blowing with propellant gas through a spinning channel as is described generally in U.S. Pat. No. 8,668,854. This process includes two phase flow of polymer or polymer solution and a pressurized propellant gas (typically air) to a thin, preferably converging channel. The channel is usually and preferably annular in configuration. It is believed that the polymer is sheared by gas flow within the thin, preferably converging channel, creating polymeric film layers on both sides of the channel. These polymeric film layers are further sheared into nanofibers by the propellant gas flow. Here again, a moving collector belt may be used and the basis weight of the nanofiber nonwoven is controlled by regulating the speed of the belt. The distance of the collector may also be used to control fineness of the nanofiber nonwoven.

Beneficially, the use of the aforementioned polyamide precursor in the melt spinning process provides for significant benefits in production rate, e.g., at least 5% greater, at least 10% greater, at least 20% greater, at least 30% greater, at least 40% greater. The improvements may be observed as an improvement in area per hour versus a conventional process, e.g., another process that does not employ the features described herein. In some cases, the production increase over a consistent period of time is improved. For example, over a given time period, e.g., one hour, of production, the disclosed process produces at least 5% more product than a conventional process or an electrospin process, e.g., at least 10% more, at least 20% more, at least 30% more, or at least 40% more.

Still yet another methodology which may be employed is melt blowing. Melt blowing involves extruding the polyamide into a relatively high velocity, typically hot, gas stream. To produce suitable nanofibers, careful selection of the orifice and capillary geometry as well as the temperature is required as is seen in: Hassan et al., J Membrane Sci., 427, 336-344, 2013 and Ellison et al., Polymer, 48 (11), 3306-3316, 2007, and, International Nonwoven Journal, Summer 2003, pg 21-28.

U.S. Pat. No. 7,300,272 (incorporated herein by reference) discloses a fiber extrusion pack for extruding molten material to form an array of nanofibers that includes a number of split distribution plates arranged in a stack such that each split distribution plate forms a layer within the fiber extrusion pack, and features on the split distribution plates form a distribution network that delivers the molten material to orifices in the fiber extrusion pack. Each of the split distribution plates includes a set of plate segments with a gap disposed between adjacent plate segments. Adjacent edges of the plate segments are shaped to form reservoirs along the gap, and sealing plugs are disposed in the reservoirs to prevent the molten material from leaking from the gaps. The sealing plugs can be formed by the molten material that leaks into the gap and collects and solidifies in the reservoirs or by placing a plugging material in the reservoirs at pack assembly. This pack can be used to make nanofibers with a melt blowing system described in the patents previously mentioned. The systems and method of U.S. Pat. No. 10,041,188 (incorporated herein by reference) are also exemplary.

In one embodiment, a process for preparing the AM/AV nonwoven polyamide structure, e.g., for use in the topcoat layer and/or the pad layer, is disclosed. The process comprising the step of forming a (precursor) polyamide (preparation of monomer solutions are well known), e.g., by preparing an aqueous monomer solution. During preparation of the precursor, a metallic compound is added (as discussed herein). In some cases, the metallic compound is added to (and dispersed in) the aqueous monomer solution. Phosphorus may also be added. In some cases, the precursor is polymerized to form a polyamide composition. The process further comprises the steps of forming polyamide fibers and forming the AM/AV polyamide fibers into a structure. In some cases, the polyamide composition is melt spun, spunbonded, electrospun, solution spun, or centrifugally spun.

A fabric can be made from the fibers by conventional means.

### EXAMPLE

Four sets of AM/AV materials (diapers) were prepared. The AM/AV materials comprised a topsheet layer and a backsheet layer with an absorbent core, and in some cases an acquisition distribution layer, configured between. The topsheet and/or ADL AM/AV layers were formed from an AM/AV composition comprising PA66, 475-625 ppm zinc (ionic), and less than 0.3 wt % processing aids, commercial stabilizers, etc. These compositions were formed, e.g., spunbond, into the layers below with the basis weights noted in Table 1.

The AM/AV diapers were tested for rewet (grams) and strikethrough (seconds) performance as described herein. The test results for set 1 are shown in Table 1. The numbers after “Avg. RW” and “Avg. ST” indicate the number of water applications.

TABLE 1
Test Results, Set 1

| | Topsheet | | | | | | | |
|---|---|---|---|---|---|---|---|---|
| | | | 34 gsm | 20 gsm | | 10 gsm | 10 gsm | 10 gsm |
| | Comp. A | Comp. B | AM/AV | AM/AV | | AM/AV | AM/AV | AM/AV |
| | ADL | | | | | | | |
| | | | | | 37 gsm | | 34 gsm | 34 gsm |
| | | | | | AM/AV | | AM/AV | AM/AV |
| Avg. RW 1 | 0.4 | 0.5 | 0.4 | 0.3 | 0.1 | 0.4 | 0.1 | 0.1 |
| Avg. RW 2 | 0.8 | 0.8 | 0.7 | 0.5 | 0.6 | 0.8 | 0.4 | 1.0 |
| Avg. RW 3 | 2.2 | 1.7 | 1.8 | 1.6 | 6.4 | 1.9 | 6.3 | 7.6 |
| Avg. RW 4 | 8.1 | 7.4 | 8.3 | 8.5 | 9.0 | 7.4 | 8.7 | 9.0 |
| Avg. ST 1 | 20 | 18 | 15 | 16 | 82 | 15 | 63 | 66 |
| Avg. ST 2 | 20 | 17 | 15 | 17 | 123 | 17 | 90 | 89 |
| Avg. ST 3 | 23 | 19 | 18 | 19 | 174 | 17 | 123 | 114 |
| Avg. ST 4 | 25 | 23 | 21 | 24 | 228 | 19 | 152 | 141 |

The test results for set 2 are shown in Table 2

TABLE 2
Test Results, Set 2

| | Topsheet | | |
|---|---|---|---|
| | | | 10 gsm |
| ADL | Comp. A | Comp. B | AM/AV |
| Avg. RW 1 | 0.1 | 0.1 | 0.1 |
| Avg. RW 2 | 1.7 | 0.8 | 0.4 |
| Avg. RW 3 | 7.7 | 6.9 | 6.2 |
| Avg. RW 4 | 9.0 | 8.9 | 8.6 |
| Avg. ST 1 | 13 | 13 | 12 |
| Avg. ST 2 | 5 | 4 | 4 |
| Avg. ST 3 | 4 | 3 | 3 |
| Avg. ST 4 | 3 | 3 | 3 |

The test results for set 3 are shown in Table 3

TABLE 3
Test Results, Set 3

| | Topsheet | | | |
|---|---|---|---|---|
| | | | | 20 gsm |
| ADL | Comp. A | Comp. B | 10 gsm AM/AV | AM/AV |
| Avg. RW 1 | 0.4 | 0.6 | 0.2 | 0.4 |
| Avg. RW 2 | 0.7 | 1.0 | 0.3 | 0.7 |
| Avg. RW 3 | 7.4 | 5.9 | 2.8 | 5.2 |
| Avg. RW 4 | 8.9 | 9.0 | 8.1 | 8.5 |
| Avg. ST 1 | 16 | 13 | 11 | 11 |
| Avg. ST 2 | 11 | 9 | 9 | 9 |
| Avg. ST 3 | 13 | 9 | 9 | 9 |
| Avg. ST 4 | 14 | 11 | 11 | 10 |

The test results for set 4 are shown in Table 4.

TABLE 4
Test Results, Set 4

Topsheet

| | | | 10 gsm | 20 gsm |
|---|---|---|---|---|
| ADL | Comp. A | Comp. B | AM/AV | AM/AV |
| Avg. RW 1 | 0.1 | 0.1 | 0.1 | 0.1 |
| Avg. RW 2 | 0.1 | 0.2 | 0.2 | 0.2 |
| Avg. RW 3 | 0.6 | 0.5 | 1.5 | 1.2 |
| Avg. RW 4 | 3.4 | 3.4 | 6.6 | 5.4 |
| Avg. ST 1 | 19 | 19 | 16 | 16 |
| Avg. ST 2 | 13 | 12 | 12 | 12 |
| Avg. ST 3 | 11 | 10 | 9 | 8 |
| Avg. ST 4 | 12 | 10 | 9 | 8 |

As shown in Tables 1-4, the disclosed AM//AV materials demonstrated good mechanical performance—on par with the comparative examples. Stated another way, surprisingly, there was no performance drop-off when employing the AM/AV layers (as opposed to the comparative layers that did not comprise the AM/AV compound and did not demonstrate advantageous AM/AV performance).

As for AM/AV performance, the 20 gsm topsheets of Set 3 were tested for Staph aureus and Klebsiella pneumoniae reduction performance via ISO 20743:2013. Additional comparative topsheets (from a commercial adult underwear product) were also tested (Comparative C). The 20 gsm topsheets comprised AM/AV compound, e.g., 475-625 ppm zinc (ionic). The comparative examples did not comprise any AM/AV compound. The averaged results are presented in Table 5.

TABLE 5
AM/AV Test Results

| Example/Comp. | Staph Log Red. (avg.) | Kleb Log Red. (avg.) |
|---|---|---|
| Set 3, 20 gsm SB | 7.5 | 8.16 |
| Set 3, Comp. B | 2.55 | 0.53 |
| Comp. C | 1.96 | 2.36 |

As shown in Table 5, the disclosed AM/AV materials not only demonstrated suitable mechanical performance, e.g., rewet and strikethrough, but advantageously also provided the synergistic AM/AV benefits described herein. For example, the working example demonstrated almost a 200% improvement over Set 3, Comp. B and almost a 300% improvement over Comp. C (along with the comparable mechanical performance).

### Embodiments

As used below, any reference to a series of embodiments is to be understood as a reference to each of those embodiments disjunctively (e.g., “Embodiments 1-4” is to be understood as “Embodiments 1, 2, 3, or 4”).

Embodiment 1 is an AM/AV material, comprising a topsheet layer comprising fibers comprising a topsheet polymer composition; a backsheet layer comprising fibers comprising a backsheet polymer composition; and an absorbent core configured therebetween; wherein the fibers of at least one of the layers, e.g., the topsheet layer, comprise an AM/AV compound, and wherein the fibers of at least one of the layers demonstrate a toilet odor reduction greater than 50%, as measured in accordance with ISO 17299-3 (2014) and/or a rewet value less than 5 g after a first water application and/or a Staph aureus efficacy log reduction greater than 2.6, as measured in accordance with ISO 20743:2013.

Embodiment 2 is the AM/AV material of embodiment 1, wherein the topsheet polymer composition comprises a polymer and an AM/AV compound.

Embodiment 3 is the AM/AV material of embodiment(s) 1 or 2, wherein the topsheet polymer composition comprises a polyamide and a zinc compound.

Embodiment 4 is the AM/AV material of embodiment(s) 1-3, wherein polymer compositions comprise a polymer comprising a thermoplastic polymer, polyester, nylon, rayon, polyamide, polyamide, poly olefin, polyolefin terephthalate, polyolefin terephthalate glycol, co-PET, or polylactic acid, or combinations thereof.

Embodiment 5 is the AM/AV material of embodiment(s) 1-4, wherein the fibers in each of the layers have an average fiber diameter from 1 micron to 50 microns.

Embodiment 6 is the AM/AV material of embodiment(s) 1-5, wherein the topsheet has a basis weight ranging from 5 gsm to 40 gsm.

Embodiment 7 is the AM/AV material of embodiment(s) 1-6, wherein the topsheet layer is meltblown, spunbond, electrospun, spunlace, or flashspun.

Embodiment 8 is the AM/AV material of embodiment(s) 1-7, wherein the topsheet layer has a thickness ranging from 25 microns to 500 microns and/or the backsheet layer has a thickness ranging from 25 microns to 500 microns.

Embodiment 9 is the AM/AV material of embodiment(s) 1-8, wherein the AM/AV material demonstrates efficacy against odor, microbials, bacteria, viruses, fungi, or parasites, or combinations thereof.

Embodiment 10 is the AM/AV material of embodiment(s) 1-9, wherein at least one of the layers demonstrate an Escherichia coli efficacy log reduction greater than 4.0, as measured in accordance with ASTM E3160 (2018).

Embodiment 11 is the AM/AV material of embodiment(s) 1-10, wherein one of more of the polymer compositions has a hygroscopy absorbance of greater than 1.5 wt. % water, based on the total weight of the polymer.

Embodiment 12 is the AM/AV material of embodiment(s) 1-11, further comprising a pad layer comprising a pad polymer composition and configured between the topsheet layer and the absorbent core.

Embodiment 13 is the AM/AV material of embodiment(s) 1-12, further comprising an acquisition distribution layer comprising an ADL polymer composition and configured between the topsheet layer and the absorbent core.

Embodiment 14 is the AM/AV material of embodiment(s) 1-13, further comprising an embossed layer comprising a embossed polymer composition and configured between the absorbent core and the backsheet layer.

Embodiment 15 is the AM/AV material of embodiment(s) 1-14, wherein the absorbent core comprises absorbent material and an AM/AV powder composition comprises an AM/AV compound and a polymer.

Embodiment 16 is an AM/AV material, comprising a topsheet layer comprising fibers comprising a topsheet polymer composition; a backsheet layer comprising fibers comprising a backsheet polymer composition; and an absorbent core configured therebetween; wherein the AM/AV material comprises an AM/AV powder composition, and wherein the AM/AV material demonstrates a toilet odor reduction greater than 50%, as measured in accordance with ISO 17299-3 (2014) and/or a rewet value less than 5 g after a first water application and/or a Staph aureus efficacy log reduction greater than 2.6, as measured in accordance with ISO 20743:2013.

Embodiment 17 is the AM/AV material of embodiment 16, wherein the AM/AV powder composition comprises an AM/AV compound and a polymer.

Embodiment 18 is the AM/AV material of embodiment 16 or 17, wherein the AM/AV powder composition comprises a polyamide and a zinc compound.

Embodiment 19 is the AM/AV material of embodiment(s) 16-18, wherein the AM/AV powder composition is dispersed in the absorbent core and/or in one of the layers.

Embodiment 20 is the AM/AV material of embodiment(s) 1-19, wherein polymer compositions comprise a polymer comprising a thermoplastic polymer, polyester, nylon, rayon, polyamide, polyamide, poly olefin, polyolefin terephthalate, polyolefin terephthalate glycol, co-PET, or polylactic acid, or combinations thereof.

Embodiment 21 is a process for making an AM/AV material, comprising the steps of: providing a topsheet layer comprising fibers comprising a topsheet polymer composition; a backsheet layer comprising fibers comprising a backsheet polymer composition; and an absorbent core; wherein the fibers of at least one of the layers comprise an AM/AV compound configuring the absorbent core between the topsheet layer and the backsheet layer; wherein the fibers of at least one of the layers demonstrate a toilet odor reduction greater than 50%, as measured in accordance with ISO 17299-3 (2014) and/or a rewet value less than 5 g after a first water application and/or a Staph aureus efficacy log reduction greater than 2.6, as measured in accordance with ISO 20743:2013.

Embodiment 22 is the process of embodiment 21, wherein none of the layers are treated to improve initial hygroscopicity and/or hydrophilicity.

We claim:

1. An AM/AV material, comprising:
a topsheet layer comprising fibers comprising a topsheet polymer composition comprising a polymer and an AM/AV compound;
a backsheet layer comprising fibers comprising a backsheet polymer composition; and
an absorbent core configured therebetween;
wherein the fibers of the topsheet layer comprise a polymer and an AM/AV compound, and
wherein the fibers of the topsheet layer demonstrate a rewet value less than 5 g after a first water application and a Staph aureus efficacy log reduction greater than 2.6, as measured in accordance with ISO 20743:2013.

2. The material of claim 1, wherein the topsheet polymer composition comprises a polyamide and a zinc compound.

3. The material of claim 1, wherein polymer compositions comprise a polymer comprising a thermoplastic polymer, polyester, nylon, rayon, polyamide, polyamide, poly olefin, polyolefin terephthalate, polyolefin terephthalate glycol, co-PET, or polylactic acid, or combinations thereof.

4. The material of claim 1, wherein the fibers in each of the layers have an average fiber diameter from 1 micron to 50 microns.

5. The material of claim 1, wherein the topsheet has a basis weight ranging from 5 gsm to 40 gsm.

6. The material of claim 1, wherein the topsheet layer is meltblown, spunbond, electrospun, spunlace, or flashspun.

7. The material of claim 1, wherein the topsheet layer has a thickness ranging from 25 microns to 500 microns and/or the backsheet layer has a thickness ranging from 25 microns to 500 microns.

8. The material of claim 1, wherein the fibers of the topsheet layer demonstrate a Klebsiella pneumoniae efficacy log reduction greater than 2.4, as measured in accordance with ISO 20743 :2013.

9. The material of claim 1, wherein the topsheet layer demonstrates an Escherichia coli efficacy log reduction greater than 4.0, as measured in accordance with ASTM E3160 (2018).

10. The material of claim 1, wherein one of more of the polymer compositions has a hygroscopy absorbance of greater than 1.5 wt. % water, based on the total weight of the polymer.

11. The material of claim 1, further comprising a pad layer comprising a pad polymer composition and configured between the topsheet layer and the absorbent core and/or an acquisition distribution layer comprising an ADL polymer composition and configured between the topsheet layer and the absorbent core.

12. The material of claim 1, further comprising an embossed layer comprising a embossed polymer composition and configured between the absorbent core and the backsheet layer.

13. The material of claim 1, wherein the absorbent core comprises absorbent material and an AM/AV powder composition comprises an AM/AV compound and a polymer.

14. An AM/AV material, comprising:
a topsheet layer comprising fibers comprising a topsheet polymer composition;
a backsheet layer comprising fibers comprising a backsheet polymer composition; and
an absorbent core configured therebetween; and
an AM/AV powder composition,
wherein the AM/AV material demonstrates a rewet value less than 5 g after a first water application and a Staph aureus efficacy log reduction greater than 2.6, as measured in accordance with ISO 20743:2013.

15. The material of claim 14, wherein the AM/AV powder composition comprises an AM/AV compound and a polymer.

16. The material of claim 14, wherein the AM/AV powder composition is dispersed in the absorbent core and/or in one of the layers.

17. The material of claim 14, wherein polymer compositions comprise a polymer comprising a thermoplastic polymer, polyester, nylon, rayon, polyamide, polyamide, poly olefin, polyolefin terephthalate, polyolefin terephthalate glycol, co-PET, or polylactic acid, or combinations thereof.

18. An AM/AV material, comprising:
a topsheet layer comprising fibers comprising a topsheet polymer composition;
a backsheet layer comprising fibers comprising a backsheet polymer composition; and
an absorbent core configured therebetween;
wherein the fibers of at least one of the layers comprise an AM/AV compound, and
wherein the fibers of at least one of the layers demonstrate a rewet value less than 5 g after a first water application and a Staph aureus efficacy log reduction greater than 2.6, as measured in accordance with ISO 20743:2013.

19. A process for making an AM/AV material, comprising the steps of:
providing a topsheet layer comprising fibers comprising a topsheet polymer composition;
a backsheet layer comprising fibers comprising a backsheet polymer composition; and an absorbent core; wherein the fibers of at least one of the layers comprise an AM/AV compound configuring the absorbent core between the topsheet layer and the backsheet layer;
wherein the fibers of at least one of the layers demonstrate a rewet value less than 5 g after a first water application and a Staph aureus efficacy log reduction greater than 2.6, as measured in accordance with ISO 20743:2013.

20. The process of claim 19, wherein none of the layers are treated to improve initial hygroscopicity and/or hydrophilicity.
